(19) United States
(12) Patent Application Publication
(10) Pub. No.: US 2025/0169557 A1
(43) Pub. Date: May 29, 2025

(54) PERSONAL PROTECTION SYSTEM AND METHOD

(71) Applicant: PABBAN DEVELOPMENT, INC., IRVINE, CA (US)

(72) Inventors: LAWRENCE J. GREEN, Santa Ana, CA (US); CARLO C. MEMITA, Laguna Hills, CA (US); CHRISTOPHER SIMBULAN, Irvine, CA (US); JACOB HERBERT, Irvine, CA (US); ALLAN SCHULTZ, Irvine, CA (US); JOSE MAURICIO GARCIA PRIETO, Irvine, CA (US); H. NICHOLAS HERBERT, Irvine, CA (US); WILLIAM KIRK, Irvine, CA (US)

(21) Appl. No.: 19/038,649

(22) Filed: Jan. 27, 2025

Related U.S. Application Data
(63) Continuation of application No. 17/909,964, filed on Sep. 7, 2022.
Application No. 17/909,964 is a continuation-in-part of application No. 16/925,770, filed on Jul. 10, 2020, now Pat. No. 11,219,254.
(60) Provisional application No. 62/989,416, filed on Mar. 13, 2020.

(51) Int. Cl. A41D 13/11; A42B 3/22; A61F 9/04

(52) U.S. Cl. CPC A41D 13/1184; A41D 13/1161; A42B 3/221; A61F 9/045; A42B 3/225

(57) ABSTRACT

A protective headgear system includes a support configured to engage the head of a user, a facial shield having an upper portion coupled to the support and a lower portion having a lower extremity, a flexible sheet coupled to the facial shield, a base carried by the support, a first microphone carried below the lower extremity of the facial shield and preferentially oriented away from the user, a second microphone carried by the base and preferentially oriented toward the user, a first audio generator capable of being sensed by at least one ear of the user, and a second audio generator including a loudspeaker carried by the base such that the loudspeaker is located below the lower extremity of the facial shield and is oriented for preferential sound energy delivery generally away from the user.

### INCORPORATION BY REFERENCE TO ANY PRIORITY DOCUMENTS

This application is a continuation of U.S. patent application Ser. No. 17/909,964, filed on Sep. 7, 2022, which is a U.S. National Stage patent application for PCT application no. PCT/US2021/022259, filed on Mar. 13, 2021, which is a Continuation-in-Part of U.S. patent application Ser. No. 16/925,770, filed on Jul. 10, 2020, now U.S. Pat. No. 11,219,254, which claims the benefit of priority to U.S. Provisional Application No. 62/989,416, filed on Mar. 13, 2020, all of which are incorporated by reference in their entirety herein for all purposes. Priority is claimed pursuant to 35 U.S.C. § 120 and 35 U.S.C. § 119.

### BACKGROUND OF THE INVENTION

### Field of the Invention

The field of the invention generally relates to personal protection systems, including, but not limited to personal environmental protections systems. The personal protections systems often include a headgear structure which is worn by an individual to protect from particulate material. The personal protection systems may provide filtered air to the user.

### SUMMARY OF THE INVENTION

In one embodiment of the present disclosure, a protective headgear system includes a support configured to engage the head of a user, a facial shield having an upper portion coupled to the support and a lower portion having a lower extremity, a flexible sheet coupled to at least the lower portion of the facial shield, the flexible sheet configured to cover at least a portion of the upper torso of the user, a base carried by the support and downwardly extending at least to a position at or adjacent the lower extremity of the facial shield, a first microphone including a first sound energy sensor and configured to generate a first signal, the first microphone carried by the base such that the first sound energy sensor is located below the lower extremity of the facial shield and is preferentially oriented away from the user, a second microphone including a second sound energy sensor and configured to generate a second signal, the second microphone carried by the base such that the second sound energy sensor is preferentially oriented toward the user, a first audio generator configured to generate a first vibration capable of being sensed by at least one ear of the user, the first vibration at least partially generated from the first signal, and a second audio generator including a loudspeaker configured to generate a second vibration at least partially generated from the second signal, the loudspeaker carried by the base such that the loudspeaker is located below the lower extremity of the facial shield and is oriented for preferential sound energy delivery generally away from the user.

In another embodiment of the present disclosure, a protective headgear system includes a support configured to engage the head of a user, a facial shield including a polymeric sheet having an upper portion configured to be coupled to the support and a lower portion having a lower extremity, wherein the upper portion includes a first hole, a second hole, and a third hole, each one of the first, second, and third holes substantially spaced from the other two of the first, second, and third holes, and wherein the first and second holes are each substantially elongated, a first hook carried on the support, wherein the first hole is configured to be engaged with first hook, a second hook carried on the support, wherein the second hole is configured to be engaged with second hook, a third hook carried on the support, wherein the third hole is configured to be engaged with third hook, wherein the first hook includes a hook portion having a maximum hook width, the first hook located on a perimeter of the support between the second and third hooks, and wherein the first hole has a first hole gap and a first hole width, the first hole width of the first hole at least about 25% greater than the maximum hook width of the hook portion of the first hook.

In yet another embodiment of the present disclosure, a protective headgear system includes a support configured to engage the head of a user, a facial shield including a polymeric sheet having an upper portion configured to be coupled to the support and a lower portion having a lower extremity, wherein the upper portion includes a first hole, a second hole, and a third hole, each one of the first, second, and third holes laterally spaced from the other two of the first, second, and third holes, a first hook carried on the support, wherein the first hole is configured to be engaged with first hook, a second hook carried on the support, wherein the second hole is configured to be engaged with second hook, a third hook carried on the support, wherein the third hole is configured to be engaged with third hook, wherein the first hook is located on a perimeter of the support between the second hook and the third hook, wherein the first hook includes a hook portion having a maximum hook width, and wherein the first hole has a lateral width greater than the maximum hook width of the hook portion of the first hook, and wherein the facial shield has a first position in relation to the support such that the first hole can be placed over the hook portion of the first hook and the second hole is not oriented to be coupled to the second hook, and wherein the first hole is laterally slidable in relation to the first hook such that the facial shield is locked to the first hook via the first hole and such that the facial shield has a second position in relation to the support, wherein the second hole is oriented to be coupled to the second hook.

In still another embodiment of the present disclosure, a facial shield for a protective headgear system including a support configured to engage the head of a user and first, second, and third hooks carried on the support, wherein the first hook is located on a perimeter of the support between the second hook and the third hook, and wherein the first hook includes a hook portion having a maximum vertical dimension and a base coupled to the support and having a vertical base thickness, includes a polymeric sheet having an upper portion configured to be coupled to the support and a lower portion having a lower extremity, wherein the upper portion includes a first hole, a second hole, and a third hole, each one of the first, second, and third holes laterally spaced from the other two of the first, second, and third holes, and the first hole including a first section having a first hole gap and a second section laterally adjacent the first section and having a second hole gap, wherein the first hole gap of the first hole is greater than or equal to the maximum vertical dimension of the first hook, and wherein the second hole gap of the first hole is greater than the vertical base thickness of the base of the first hook and is less than the maximum vertical dimension of the first hook.

In yet another embodiment of the present disclosure, a method for donning a protective headgear system including a support configured to engage the head of a user and first, second, and third hooks carried on the support, wherein the first hook is located on a perimeter of the support between the second hook and the third hook, and wherein the first hook includes a hook portion having a maximum vertical dimension and a base coupled to the support and having a vertical base thickness, includes providing a polymeric sheet having an upper portion configured to be coupled to the support and a lower portion having a lower extremity, wherein the upper portion includes a first hole, a second hole, and a third hole, each one of the first, second, and third holes laterally spaced from the other two of the first, second, and third holes, wherein the first hole includes a first section having a first hole gap and a second section laterally adjacent the first section and having a second hole gap, wherein the first hole gap of the first hole is greater than or equal to the maximum vertical dimension of the first hook, and wherein the second hole gap of the first hole is greater than the vertical base thickness of the base of the first hook and is less than the maximum vertical dimension of the first hook, placing the first hole over the first hook, sliding the polymeric sheet in relation to the support to lock the first hole to the first hook, attaching the second hook to the second hole, and attaching the third hook to the third hole.

### BRIEF DESCRIPTION OF THE DRAWINGS

FIG. 1 is a perspective view of a personal protection system in place on a user, according to an embodiment of the present disclosure.

FIG. 2 is an exploded view of the personal protection system of FIG. 1.

FIG. 3 is a front view of the personal protection system of FIG. 1.

FIG. 4 is a side view of a center clip of the personal protection system of FIG. 3.

FIG. 5 is a perspective view of a side clip of the personal protection system of FIG. 3.

FIG. 6 is a facial shield configured to engage with the clips of the personal protection system of FIG. 3, according to an embodiment of the present disclosure.

FIG. 7 is a facial shield in a first position in relation to a center clip, according to an embodiment of the present disclosure.

FIG. 8 is the facial shield of FIG. 7 is a second position in second, locked position in relation to the center clip.

FIG. 9 is a flow chart for a method for attaching a facial shield to a head support, according to an embodiment of the present disclosure.

FIG. 10 is a cross-sectional view of a facial shield being assembled onto a center clip, according to an embodiment of the present disclosure.

FIG. 11 is a cross-sectional view of the facial shield in assembled configuration on the center clip, according to an embodiment of the present disclosure.

FIG. 12 is a cross-sectional view of the facial shield being assembled onto a side clip, according to an embodiment of the present disclosure.

FIG. 13 is a cross-sectional view of the facial shield further being assembled onto the side clip, according to an embodiment of the present disclosure.

FIG. 14 is a cross-sectional view of the facial shield in assembled configuration on the side clip.

FIG. 15 is a perspective view of the facial shield being attached to the center clip, according to an embodiment of the present disclosure.

FIG. 16 is a detail perspective view of the facial shield being locked to the center clip, according to an embodiment of the present disclosure.

FIG. 17 is a perspective view of the facial shield being adjusted prior to assembly to a first side clip, according to an embodiment of the present disclosure.

FIG. 18 is a perspective view of the facial shield being attached to the first side clip, according to an embodiment of the present disclosure.

FIG. 19 is a perspective view of the facial shield being locked to the first side clip, according to an embodiment of the present disclosure.

FIG. 20 is a perspective view of the facial shield being adjusted prior to assembly to a second side clip, according to an embodiment of the present disclosure.

FIG. 21 is a perspective view of the facial shield being attached to the second side clip, according to an embodiment of the present disclosure.

FIG. 22 is a perspective view of the facial shield being locked to the second side clip, according to an embodiment of the present disclosure.

FIG. 23 is a front view of the personal protection system of FIG. 3 with the facial shield coupled to the head support.

FIG. 24 is a front view of a personal protection system according to an embodiment of the present disclosure.

FIG. 25 is a perspective view of a side clip of the personal protection system of FIG. 24.

FIG. 26 is a perspective view of a center clip of the personal protection system of FIG. 24.

FIG. 27 is a facial shield configured to engage with the clips of the personal protection system of FIG. 24, according to an embodiment of the present disclosure.

FIG. 28 is a perspective view of a personal protection system incorporating the facial shield and cuff of FIG. 27, according to an embodiment of the present disclosure.

FIG. 29 is an underside view of the personal protection system of FIG. 28.

FIG. 30 is a perspective view of an alternative personal protection system in place on a user, according to an embodiment of the present disclosure.

FIG. 31 is a hook strip assembly of the personal protection system of FIG. 30.

FIG. 32 is a perspective view of a personal protection system being assembled on a user, according to an embodiment of the present disclosure.

FIG. 33 is a perspective view of the personal protection system of FIG. 32 in place on a user, according to an embodiment of the present disclosure.

FIG. 34 is a detail view of a center hook of the personal protection system of FIG. 33.

FIG. 35 is a perspective view of an extender for the personal protection system of FIG. 33.

FIG. 36 is a front view of the personal protection system, according to an embodiment of the present disclosure.

FIG. 37 is a perspective view of the center hook of the personal protection system of FIG. 36.

FIG. 38 is a side view of the center hook of the personal protection system of FIG. 36.

FIG. 39 is a facial shield configured to engage with the clips of the personal protection system of FIG. 36, according to an embodiment of the present disclosure.

FIG. 40 is a perspective view of a personal protection system in place on a user, according to an embodiment of the present disclosure.

FIG. 41 is a perspective view of a personal protection system in place on a user, according to an embodiment of the present disclosure.

FIG. 42 is a perspective view of the personal protection system of FIG. 40 in place on a user.

FIG. 43 is a perspective view of the personal protection system of FIG. 40.

FIG. 44 is a perspective view of an alternative personal protection system in place on a user according to an embodiment of the present disclosure.

FIG. 45 is a perspective view of the personal protection system of FIG. 44.

FIG. 46 is a cross-sectional view of a portion of the personal protection system of FIGS. 42 and 43.

FIG. 47 is a bottom view of a helmet of a personal protection system, according to an alternative embodiment of the present disclosure.

FIG. 48 is a perspective view of a personal protection system in place on a user, according to an embodiment of the present disclosure.

FIG. 49 is a side view of a personal protection system in place on a user, according to an embodiment of the present disclosure.

FIG. 50 is a perspective view of a front of the personal protection system of FIG. 49.

FIG. 51 is a detail view of an upper front portion of the personal protection system of FIG. 49.

FIG. 52 is a detail view of an upper front portion of the personal protection system of FIG. 49.

FIG. 53 is a front view of a protective hood according to an embodiment of the present disclosure.

FIG. 54 is a front view of the protective hood of FIG. 53 in place on a user and a gown being placed thereon, according to an embodiment of the present disclosure.

FIG. 55 is a front view of the protective hood and gown of FIG. 54 fully seated upon the user.

FIG. 56 is a perspective view of a personal protection system according to an embodiment of the present disclosure.

FIG. 57 is a front view of the personal protection system of FIG. 56 during a first gowning step.

FIG. 58 is a front view of the personal protection system of FIG. 56 after the first gowning step.

FIG. 59 is a perspective view of a personal protection system according to an embodiment of the present disclosure.

FIG. 60 is a front view of the personal protection system of FIG. 59 during a first gowning step.

FIG. 61 is a front view of the personal protection system of FIG. 59 after the first gowning step.

FIG. 62 is a perspective view of a personal protection system in place on a user, according to an embodiment of the present disclosure.

FIG. 63 is a partial cross-sectional view of a hood of the personal protection of FIG. 63 with a stethoscope in place.

FIG. 64 is a cross-sectional view of a helmet, according to an embodiment of the present disclosure.

FIG. 65 is a cross-sectional view of a helmet, according to an embodiment of the present disclosure.

FIG. 66 is a perspective view of a personal protection system in place on a user, according to an embodiment of the present disclosure.

FIG. 67 is a partially exploded view of the personal protection system of FIG. 66.

FIG. 68 is a perspective view of a personal protection system in place on a user, according to an embodiment of the present disclosure.

FIG. 69 is a perspective view of a personal protection system in place on a user, according to an embodiment of the present disclosure.

### DETAILED DESCRIPTION OF THE ILLUSTRATED EMBODIMENTS

There are several types of air flow, filtration and protective systems which are known in the art. Several types of such systems are currently available on the market for use in surgical arenas, in “clean room” environments, or in hazardous/contaminated environments.

Some of the existing systems include hoods, gowns, filters, and the like. In some instances, the air filters are built into the helmet structure. Known units frequently include external sources of air such as gas cylinders, air lines or the like which are connected to the helmet structure by tubes, hoses or the like. Currently available lens/facial seal combinations, sometimes known as loose fitting hoods, are expensive to manufacture due to the geometries required for the facial seal to attach to the lens which is curved in a plane perpendicular to the seal to the face/head of the wearer. Improvements described herein related to the interface between facial shields and headgear (helmets, etc.) accommodate clean or sterile donning techniques, and improve the overall ease of donning the protective equipment and garments. In sterile procedures, any improvements that lower or minimize contact, or decomplicate the donning steps can significantly improve the likelihood of achievement and maintenance of sterility. These improvements can also minimize unnecessary contamination to other sites. The systems described herein may also be utilized for general healthcare use or general laboratory use, as well as in surgery, medical procedure, or dental use. The systems may comprise PAPR (Powered Air Purifying Respirator) systems comprising a blower with a motor, but may also comprise non-powered systems, including physical or acoustical protection systems, such as those used in construction or airport or raceway communication and/or protection. The systems described herein may also be utilized in general PPE (personal protective equipment).

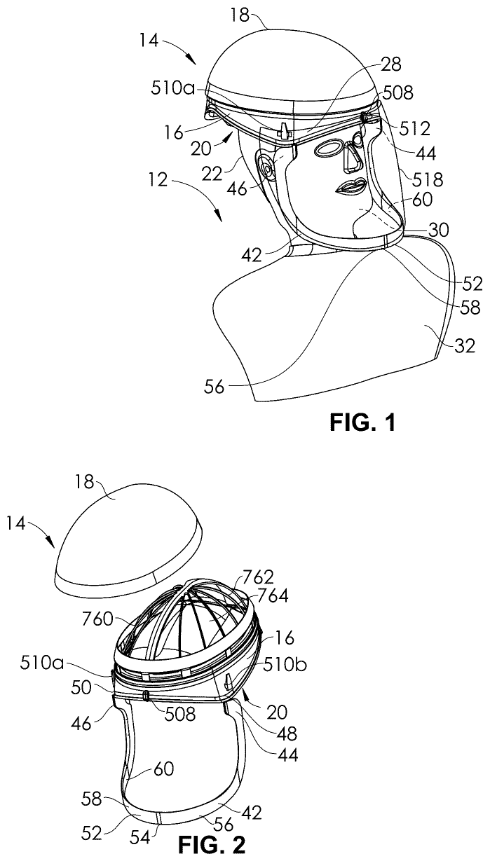

FIG. 1 illustrates a user 12 wearing a personal environmental protection system 14. The user 12 may be a surgeon, physician, or another medical personnel participating in a surgery or other medical procedure on a patient that has an ailment that is contagious in an otherwise standard, unprotected environment. This may also include any hospital or medical facility environment during an epidemic or pandemic that does or does not include the patient as a source of infection, such as the 2020 COVID-19 pandemic. The personal environmental protection system 14 comprises a head support 16 having a top portion 18 and a concave underside 20 configured to engage with the head 22 of the user 12. The head support 16 may comprise a helmet or other type of headgear that is securely and comfortably maintainable on the head 22 of the user 12 during an entire medical procedure, to avoid the need for constant removal, adjustment or replacement. A facial shield 518 (or lens) comprising a substantially optically clear material includes a lower extremity 26 configured to extend below the face 30 of the user 12, and an upper portion 28 configured to be coupled to the head support 16. The user 12 is able to clearly view through the facial shield 518 while the head support 16 is engaged with the head 22 of the user 12. Thus, upon movement of the head 22 of the user 12, the head support 16 maintains the facial shield 518 in front of the face 30 of the user 12. In some embodiments, a gown 32 or toga may be used by the user 12 to protect some, most, or all of the body of the user 12 below the head and neck, or at least to cover the upper torso of the user 12. In some embodiments, the gown 32 may couple directly to the facial shield 518, the head support 16, or to both the facial shield 518 and the head support 16. The personal environmental protection system 14 may be configured to substantially control the breathing environment of the use 12 via air filtration, inflow, and/or outflow, and may utilize the operative elements for air filtration, inflow, and/or outflow in any of the embodiments described in U.S. Pat. No. 8,302,599 to Green issued Nov. 6, 2012, and entitled “Protective Headgear System with Filter Protector,” which is incorporated herein by reference in its entirety for all purposes. The personal environmental protection system 14 may additionally or alternatively utilize the operative elements for air filtration, inflow, and/or outflow in any of the embodiments described in U.S. Pat. No. 8,453,262 to Green issued Jun. 4, 2013, and entitled “Personal Environmental Protection Apparatus,” which is incorporated herein by reference in its entirety for all purposes.

The facial shield 518 may comprise a sheet comprising high clarity polymer such as polyethylene terephthalate glycol (PETG), polyethylene terephthalate (PET), or other polyesters or polyester copolymers, or acrylic, or polycarbonate, such that it can provide a relatively thin but tough barrier that does not significantly impede the vision of the user 12. In some embodiments, the facial shield 518 may be configured to substantially control the breathing environment of the use 12 via air filtration, inflow, and/or outflow, and may utilize the operative elements for air filtration, inflow, and/or outflow in any of the embodiments has a permanent concave shape toward the user 12 and a permanent convex shape away from the user 12. In FIG. 1, however, the facial shield 518 comprises a flat flexible sheet that can be produced by die cutting or other rapid processes. The facial shield 518 is flexible and may conform to a variety of curves, such as the curve required to mate with the head support 16. In some embodiments, the facial shield 518 may comprise polycarbonate having a thickness of between about 0.010 inch and about 0.020 inch, or between about 0.012 inch and about 0.018 inch, or between about 0.014 inch and about 0.016 inch. In some embodiments, the facial shield 518 may comprise PET having a thickness of between about 0.004 inch and about 0.012 inch, or between about 0.006 inch and about 0.010 inch, or between about 0.007 inch and about 0.009 inch.

The gown 32 may comprise a number of different materials and configurations. Materials for the gown 32 may include a tri-laminate comprising a film held between two layers of non-woven plastic fabric. Bi-laminate materials are also possible, such as a material comprising a film layer and a non-woven plastic fabric. In some embodiments, the non-woven layer or layers may comprise a cellulose. In some embodiments, the non-woven layer may comprise spun materials such as spunbonded high density polyethylene (e.g., Tyvek®, a trademark of DuPont de Nemours, Inc.). In one embodiment a spunbond meltblown spunbond, commonly known as SMS, may be used, and comprises a tri-laminate non-woven fabric comprising a top layer of spunbond polypropylene, a middle layer of meltblown polypropylene and a bottom layer of spunbond polypropylene. In other embodiments, one or more of the non-woven layers may be replaced by a woven layer.

A chin bar 42 has a first end 44 connected to a first portion 48 of the head support 16 and a second end 46 connected to a second portion 50 the head support 16. The chin bar 42 may comprise a rigid polymeric material such as polyamide, for example nylon 6, or polycarbonate. The substantial rigidity allows one of the dimensions of the chin bar 42 to be relatively thin, such that it has a generally rectangular cross-section. The chin bar 42 may alternatively comprise high-density polyethylene or polypropylene, though the thin dimension would likely be thickened somewhat to maintain sufficient stiffness and resistance to extreme bending. The chin bar 42 extends between the first end 44 and the second end 46 in a generally U-shape 52 having a lower apex 54. The lower apex 54 is located at the center of a lower portion 56 of the chin bar 42 having an externally-facing surface 58 and an internally-facing surface 60.

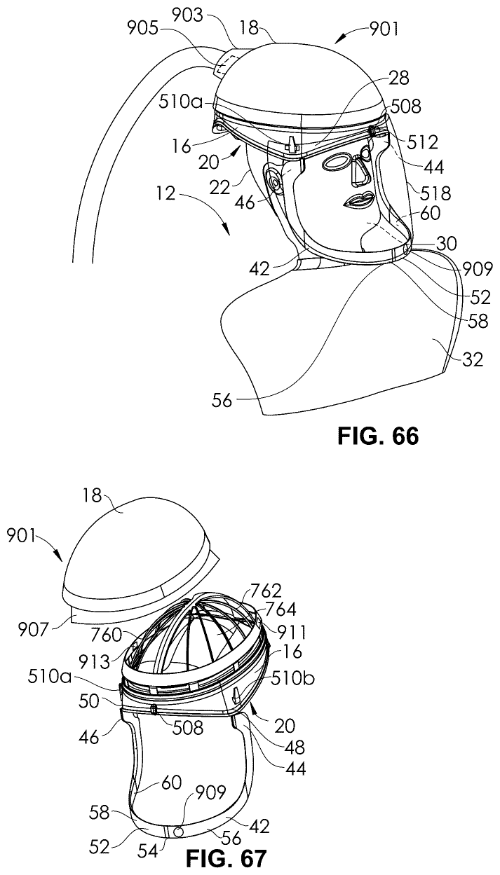

FIG. 2 illustrates an exploded view of the personal environmental protection system 14. A filter frame 760 is carried on the head support 16 and includes a series of ribs 762 defining spaces 764 between the ribs 762 configured to allow the circulation of filtered air. A fan (see blower 903 in FIG. 66) is configured to cause the flow of air within the system 14. A filter cartridge/hood assembly (see filter cartridge 907 in FIG. 67) comprises a filter cartridge within a hood, and is configured to sealably seat on the filter frame 760.

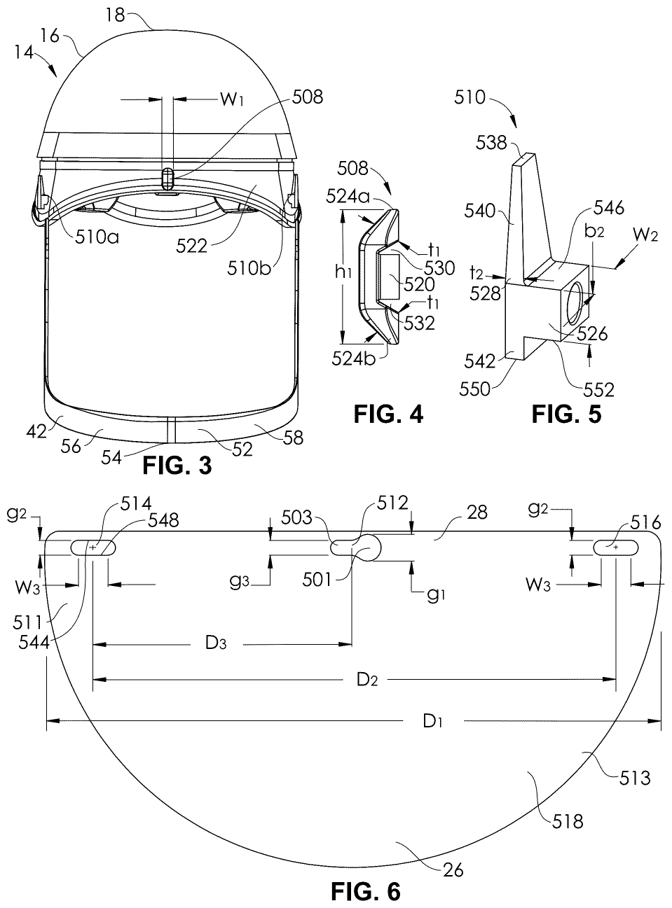

Turning to FIG. 3, the head support 16 carries a center hook 508 and two side hooks 510a, 510b. The hooks 508, 510a, 510b are configured to engage holes 512, 514, 516 of the facial shield 518, illustrated in FIG. 6. The facial shield 518 can comprise a clear sheet of the material previously described herein. The center hook 508 (FIG. 4) has a base 520 configured to couple to an outer surface 522 of the head support 16, and a hook portion 524 comprising an upper hook portion 524a and a lower hook portion 524b, and having a height h₁ and a maximum width W₁. Each of the side hooks 510a, 510b (side hook 510, FIG. 5) has a base 526 configured to couple to the outer surface 522 of the head support 16, and a hook portion 528 having a maximum width W₂. Holes 514, 516 each have a width W₃ that is greater than the maximum width W₂. Holes 514, 516 each have a gap g₂ that is greater than the base height b₂.

The upper hook portion 524a and the lower hook portion 524b of the center hook 508 each have a maximum thickness t₁. The hook portion 528 of the side hooks 510 comprises an upper hook portion 540 and a lower hook portion 542. The upper hook portion 540 of the side hooks 510 has a maximum thickness t₂. Hole 512 comprises a first section 501 and a second section 503, adjacent the first section 501. Any of the holes 512, 514, 516 described herein may be formed as part of a die-cutting process, or may be added by use of a punch tool.

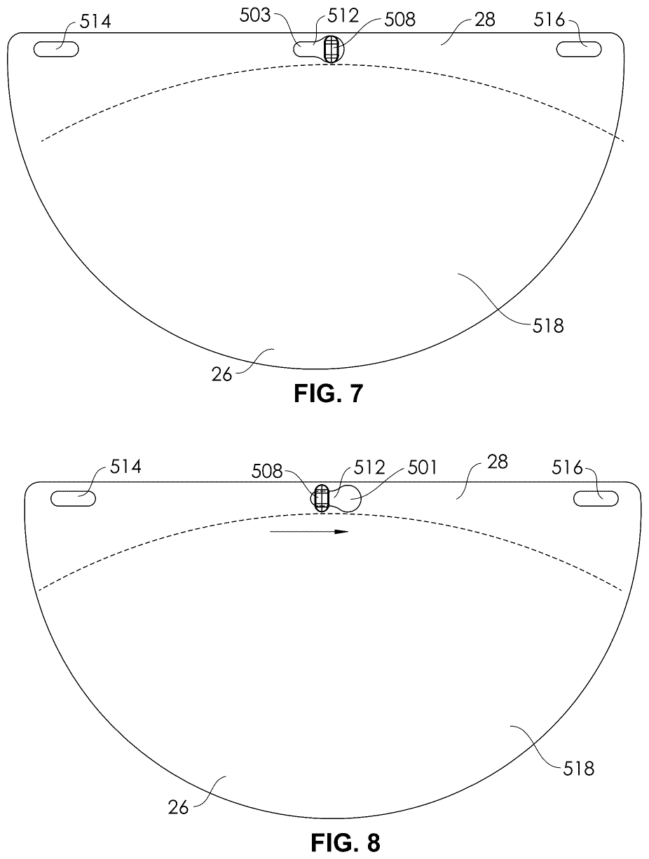

Turning to FIG. 7, the first section 501 of hole 512 comprises a circular shape having a maximum gap g₁ that is at least slightly greater than the height h₁ of the center hook 508. Thus, as shown in FIG. 7, the first section 501 of the hole 512 of the facial shield 518 may be placed completely over center hook 508. By then sliding the facial shield 518 in relation to the center hook 508 (arrow, FIG. 8), upper border 505 and lower border 507 are slid underneath the upper hook portion 524a and the lower hook portion 524b, respectively, thus locking the center hook 508 to the facial shield 518, via the hole 512. Holes 514, 516 each have a gap g₂ that is at least slightly greater than the maximum thickness t₂ of the upper hook portion 540 of the side hooks 510, thus allowing each hole 514, 516 to fit completely over each side hook 510a, 510b. Center hook 508 has an upper recess 530 and a lower recess 532 (FIG. 4), between the base 520 and the upper and lower hook portions 524a, 524b, respectively.

To attach the facial shield 518 to the head support 16, the hole 512 is first inserted over the center hook 508, as in FIG. 7, and then moved in the direction of the arrow in FIG. 8, so that the hole 512 slides and locks to the center hook 508. The second section 503 of the hole 512 has a gap g₃ that is greater than the diameter of the base 520. In some embodiments, the base 520 may be non-circular, and thus the gap g₃ is greater than a vertical height of the base 520. The second section 503 of the hole 512 now straddles the base 526. As will be shown, with the first section 501 of the hole 512 around the center hook 508, as in FIG. 7, and the facial shield 518 wrapped around the outer surface 522 (perimeter) of the head support 16, the hole 514 does not align with the side hook 510a, and the hole 516 does not align with the side hook 510b. However, after the facial shield 518 has been locked in to the position of FIG. 8, with the second section 503 of the hole 512 straddling the base 526, the hole 514 aligns with the side hook 510a and the hole 516 aligns with the side hook 510b. Thus, a user 12 or a support personnel may first lock the facial shield 518 to the center hook 508, and then, with the facial shield 518 securely coupled and also balanced around the center hook 508, the user 12 or support personnel may proceed to fully attach the facial shield 518 to the side hooks 510a, 510b.

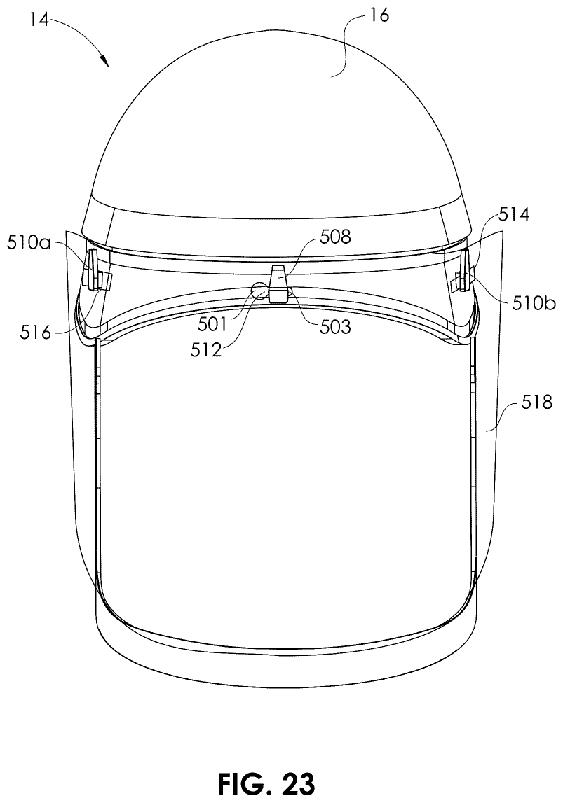

To attach the hole 514 to the side hook 510a, the hole 514 is inserted over the end 538 (FIG. 5) of the upper hook portion 540 of the hook portion 528 of the hook 510a and the right side 511 of the facial shield 518 is pulled down over the upper hook portion 540 toward the base 526. An upper edge 544 of the hole 514 is forced against an upper ledge 546 of the base 526, and the right side 511 of the facial shield 518 is forced further downward, slightly opening the gap g₂, such that a lower edge 548 of the hole 514 clears a bottom face 550 of a lower hook portion 542 of the hook portion 528. When the hole 514 is released, and loses its slight deformation, the lower edge 548 of the hole 514 is snapped into its locked, rest position, above the bottom face 550, but below a lower ledge 552 of the base 526. The hole 514 is thus locked both in relation to the upper hook portion 540 and the lower hook portion 542. The facial shield 518 thus extends substantially vertically downward at its right side, as well as at its center. If needed or desired, the facial shield 518 can now be moved slightly to the left or right (e.g., by pulling on the left side 513 or the right side 511 of the facial shield 518), with the hole 514 sliding (though still in a locked position) along the side hook 510a and with the second section 503 of the hole 512 sliding (though remaining in a locked position) along the base 520 of the center hook 508. The total width of the holes 512, 514, 516 in relation to the hooks 508, 510a, 510b determines how much lateral play there is in the locked condition. The snapping process of the hole 514 and the side hook 510a can be repeated with the hole 516 and the side hook 510b on the left side 513 of the facial shield 518. Once all holes 512, 514, 516 have been coupled to the hooks 508, 510a, 510b, the facial shield 518 is now securely in place on the head support 16, with a few quick and simple snap and slide actions, as shown in FIG. 23. Furthermore, the facial shield 518 can be attached to the head support 16 without the need for the user or support personnel (whichever is assembling the equipment) to touch the head support 16 in any way. This can significantly increase the likelihood of effective sterility. Oftentimes, the head support 16 is a reusable item while the facial shield 518 is supplied sterile. Though the head support 16 may be cleanable and may even be sterilizable, the avoidance of contact can assure a higher probability of a sterile procedure. In overtaxed, overwhelmed emergency rooms or surgical suites, either during pandemics or during out-of-control war or disaster situations, simplicity in setup can be important. Otherwise, mistakes can easily be made, with loss of sterility as one possible outcome.

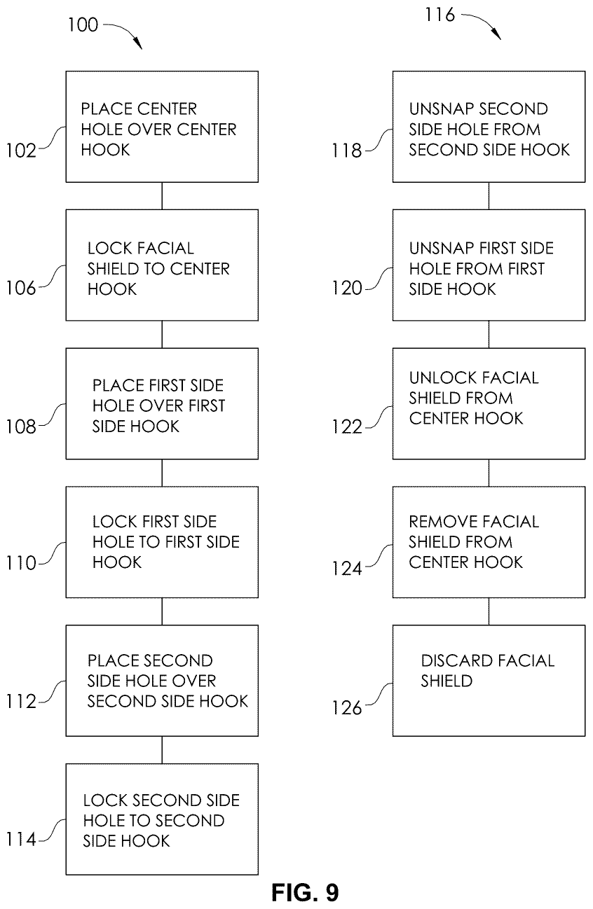

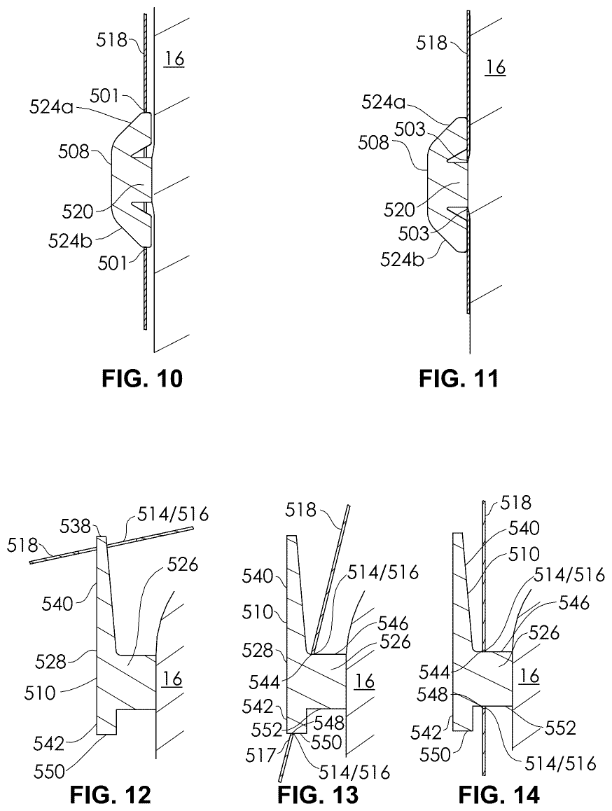

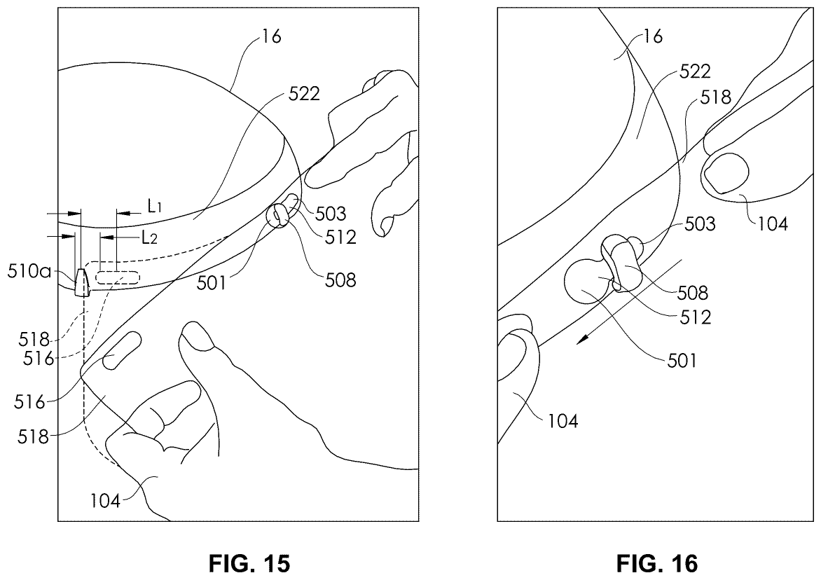

FIG. 9 illustrates a method 100 for attaching the facial shield 518 to the head support 16. In step 102 an assembler 104 (FIG. 15), who may be a user or a support personnel, aligns the first section 501 of the hole 512 of the facial shield 518 over the center hook 508. Note that the first section 501 and the second section 503 are oriented right-to-left in FIG. 15, while they are shown left-to-right in FIG. 6. The facial shield 518 may reversible, such that it can be oriented on the head support 16 in either manner. In other embodiments, the facial shield 518 may have a non-reflective coating on only one side, and may be reversible, or non-reversible. In other embodiments, the facial shield 518 may have one or more clear peelable adhesively-attached coverings on the outer surface, which can be removed if soiled or contaminated, e.g., by splashing, or scratching. Turning to FIG. 10, the diameter (gap g₁) of the first section 501 of the hole 512 is shown just clearing the height h₁ of the center hook 508 during step 102. This position is also shown in FIG. 7. In some embodiments, the gap g₁ is at least slightly larger than the height h₁ such that the first section 501 can clear the center hook 508, as the facial shield 518 is placed over it. In some embodiments, the gap g₁ (FIG. 6) is the same dimension as the height h₁, or slightly smaller than the height h₁, such that the facial shield 518 can be forced over the center hook 508 without great effort from the assembler 104.

In step 106, the assembler 104 locks the facial shield 518 to the head support 16 by sliding the head support to the left (arrow), as shown in FIG. 16, into the position shown in FIG. 8. The second section 503 of the hole 512 is now oriented around the base 520 of the center hook 508. The center hook 508, thus, locks the facial shield 518 to the head support 16, because the gap g₃ (FIG. 6) is less than the height h₁. This can be seen in more detail in FIG. 11. Though the assembler 104 in FIGS. 15-22 is shown facing the outside of the head support 16, a user having the head support 16 upon their head may perform the same steps, for example, while looking at themself in a mirror or video monitor. In the position shown in FIG. 15 and FIG. 7, the hole 516 cannot be placed over the side hook 510a (see dashed lines in FIG. 15) when the facial shield 518 is wrapped around the outer surface 522 of the head support 16. Not only are the centers of the side hook 510a and the hole 516 (dashed) offset by a distance L₁, but the entirety of the hole 516 is offset from the entirety of the side hook 510a by a distance L₂. However, when the facial shield 518 is slid into the position shown in FIGS. 16-18, the hole 516 can be placed over the side hook 510a. Thus, it is not possible to assemble hole 516 onto the side hook 510a before assembling and locking the hole 512 onto the center hook 508. The hole 512 must be attached and locked to center hook 508 first. Thus, it is assured that during the initial locked securement of the facial shield 518 to the head support 16, the facial shield 518 is significantly centered and balanced, with no portion of it able to significantly hang too low, where it may be damaged, soiled, or contaminated. This will be further described in the following steps.

In one particular embodiment: the distance D₁ is between about 12.5 inches and about 18.5 inches, or between about 14 inches and about 17 inches, or between about 14.5 inches and about 16.5 inches, or about 15.5 inches; the distance D₂, between the horizontal center of hole 514 and the horizontal center of hole 516, is between about 11 inches and about 15 inches, or between about 12 inches and about 14 inches, or between about 12.5 inches and about 13.5 inches, or between about 12.75 inches and about 13.25 inches, or about 13 inches; the gap g₁ is between about 0.60 inch and about 0.75 inch, or about 0.675 inch, and is formed by a hole having a diameter of between about 0.60 inch and about 0.75 inch, or about 0.675 inch, or greater; the gap g₂ is between about 0.225 inch and about 0.680 inch, or between about 0.300 inch and about 0.450 inch, or between about 0.325 inch and about 0.425 inch, or about 0.375 inch; the gap g₃ is between about 0.325 inch and about 0.425 inch, or about 0.375 inch; the width W₁ is between about 0.30 inch and about 0.40 inch, or about 0.35 inch; the width W₂ is between about 0.35 inch and about 0.45 inch, or about 0.40 inch; the width W₃ is between about 0.70 inch and about 0.80 inch, or about 0.75 inch; the height h₁ is between about 0.61 inch and about 0.74 inch, or between about 0.65 inch and about 0.71 inch, or about 0.68 inch; the thickness t₁ is between about 0.10 inch and about 0.18 inch, or between about 0.12 inch and about 0.16 inch, or about 0.14 inch; the thickness t₂ is between about 0.06 inch and about 0.14 inch, or between about 0.08 inch and about 0.12 inch, or about 0.10 inch; the base height b₂ is between about 0.225 inch and about 0.325 inch, or between about 0.250 inch and about 0.300 inch, or about 0.275 inch; the distance D₃, between the horizontal center of hole 512, approximately where section 501 transitions to section 503, and the horizontal center of hole 514, is between about 6.0 inches and about 7.0 inches, or between about 6.25 inches and about 6.75 inches, or about 6.50 inches. In other embodiments, the gap g₁ is greater than or equal to 0.68 inch.

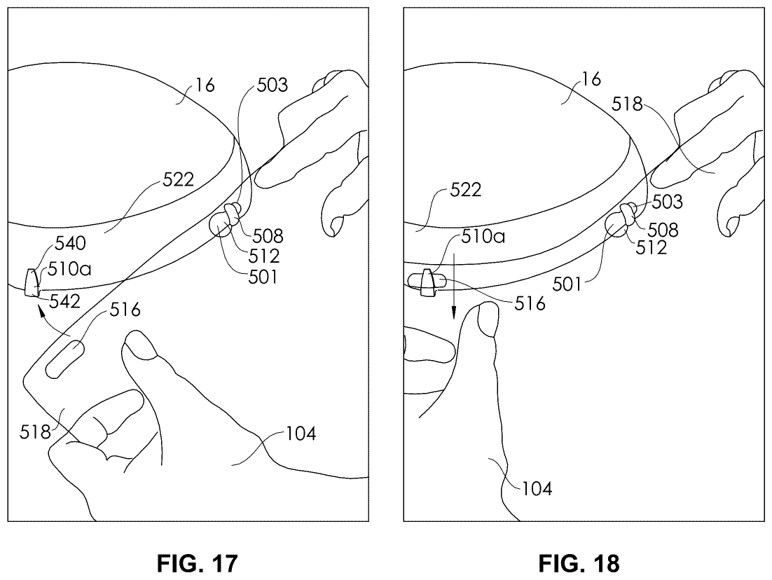

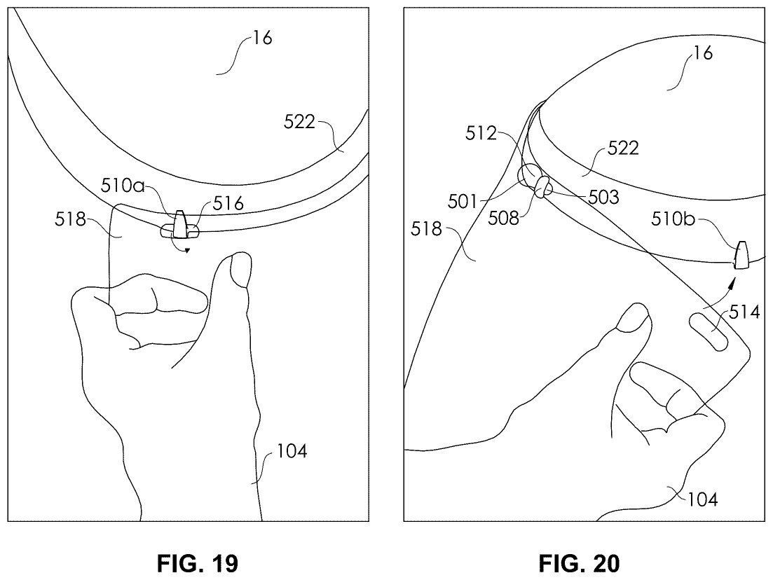

In step 108, the assembler 104 wraps the facial shield 518 around the outer surface 522 of the head support 16 and places the hole 516 over the upper hook portion 540 of the side hook 510a, as shown in FIG. 17. As shown in detail in FIG. 12, the assembler 104 passes the hole 516 over the end 538 and onto the upper hook portion 540 of the hook portion 528 of the hook 510a. In step 110, the assembler 104 pulls the facial shield 518 downward (arrow, FIG. 18) to force a lower margin 517 of material surrounding the hole 516 over the bottom face 550 of a lower hook portion 542 of the hook portion 528, as shown in FIG. 13 and FIG. 19. An upper edge 544 of the hole 516 is forced against an upper ledge 546 of the base 526, and the right side of the facial shield 518 is forced further downward, slightly opening the gap g₂ (FIG. 6), such that a lower edge 548 of the hole 516 clears a bottom face 550 of a lower hook portion 542 of the hook portion 528. Turning to FIG. 14, when the hole 516 is released, and loses its slight deformation, the lower edge 548 of the hole 516 is snapped into its locked, rest position, above the bottom face 550, but below a lower ledge 552 of the base 526. The hole 516 is thus locked both in relation to the upper hook portion 540 and the lower hook portion 542.

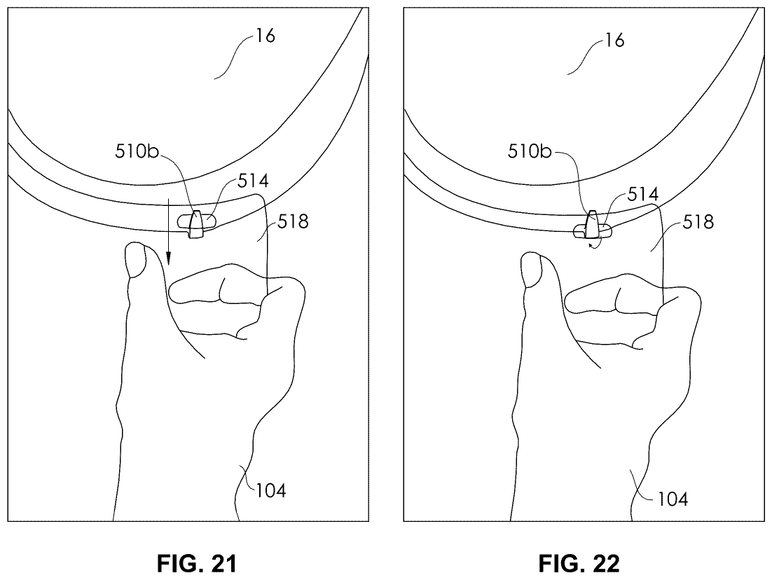

In step 112, the assembler 104 wraps the opposite side of the facial shield 518 around the outer surface 522 of the head support 16 and places the hole 514 over the upper hook portion 540 of the side hook 510b, as shown in FIG. 20. As shown in detail in FIG. 12, the assembler 104 passes the hole 514 over the end 538 and onto the upper hook portion 540 of the hook portion 528 of the hook 510b. In step 114, the assembler 104 pulls the facial shield 518 downward (arrow, FIG. 21) to force a lower margin 517 of material surrounding the hole 514 over the bottom face 550 of a lower hook portion 542 of the hook portion 528, as shown in FIG. 13 and FIG. 22. An upper edge 544 of the hole 514 is forced against an upper ledge 546 of the base 526, and the right side of the facial shield 518 is forced further downward, slightly opening the gap g₂ (FIG. 6), such that a lower edge 548 of the hole 514 clears a bottom face 550 of a lower hook portion 542 of the hook portion 528. Turning to FIG. 14, when the hole 514 is released, and loses its slight deformation, the lower edge 548 of the hole 514 is snapped into its locked, rest position, above the bottom face 550, but below a lower ledge 552 of the base 526. The hole 514 is thus locked both in relation to the upper hook portion 540 and the lower hook portion 542. FIG. 23 illustrates the facial shield 518 fully assembled onto the head support 16. As shown in FIGS. 15-22, the facial shield 518 can be easily and securely attached to the head support 16 without touching the head support 16 at all.

FIG. 9 further illustrates a method 116 for removal of the facial shield 518 from the head support 16. In step 118, the user 104 unsnaps the hole 514 and removes the facial shield 518 from the side hook 510b. In step 120, the user 104 unsnaps the hole 516 and removes the facial shield 518 from the side hook 510a. In step 122, the user slides the facial shield 518 in relation to the head support 16 to unlock the hole 512 from the center hook 508, thus placing the facial shield 518 into the position of FIGS. 7, 10, and 15. In step 124, the user 104 completely removes the facial shield 518 from the head support 16. In step 126, the user 14 discards the facial shield 126. In some cases, the user 104 may also choose to discard the head support 16.

In other embodiments, any one or more of the holes 512, 514, 516 may be vertically elongated instead of horizontally elongated. One or more of the holes 512, 514, 516 may even be diagonally elongated. The facial shield 518 can be produced from standard sheet material and can be mass produced by a die cutting process thus significantly lowering the cost in comparison with traditional curved, single-shape facial shields. The locking and unlocking utilizing the hooks 508, 510 and the holes 512, 514, 516 provides quick yet secure assembly and quick removal and cleaning or disposal.

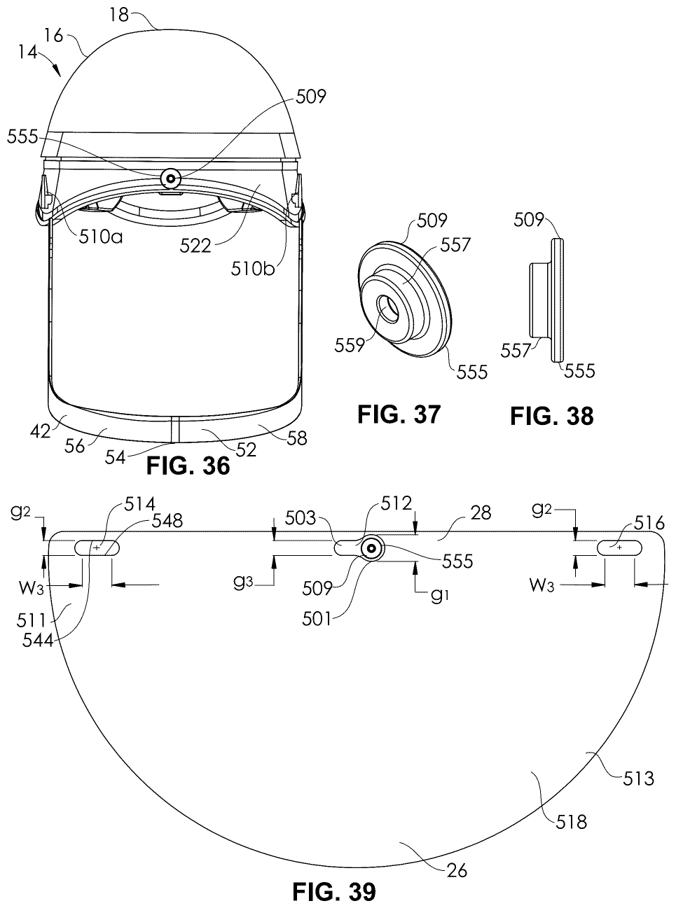

An alternative hook 509 may be used in place of hook 508, as shown in FIGS. 36-39. The hook 509 includes a proximal hole 559 configured to attach it to the head support 16. The hook 508 may be configured to snap onto a fastener on the head support 16, or screw onto a fastener on the head support 16, or slide onto a fastener on the head support 16, or be adhesively or epoxy bonded to the head support 16. The hook 509 comprises a circular shape having a first circular section 555 having a diameter that is less than the maximum gap g₁ of the first section 501 of hole 512. Hook 509 further comprises a second circular section 557 having a diameter that is less than the gap g₃ of the second section 503 of hole 512. The diameter of the first circular section 555 is greater than the gap g₃ of the second section 503 of hole 512. Thus, the hook 509 maintains similar locking/unlocking functionality with the hole 512 of the facial shield 518.

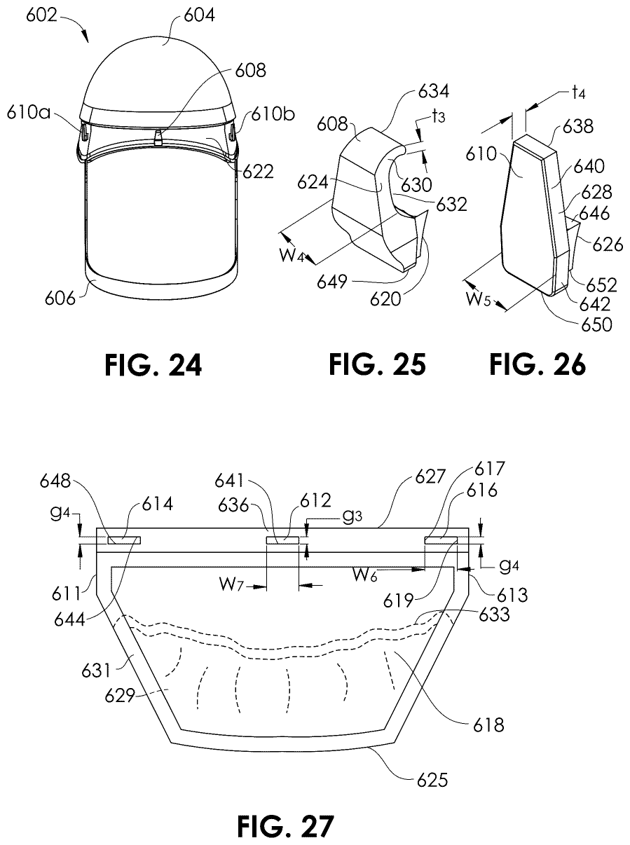

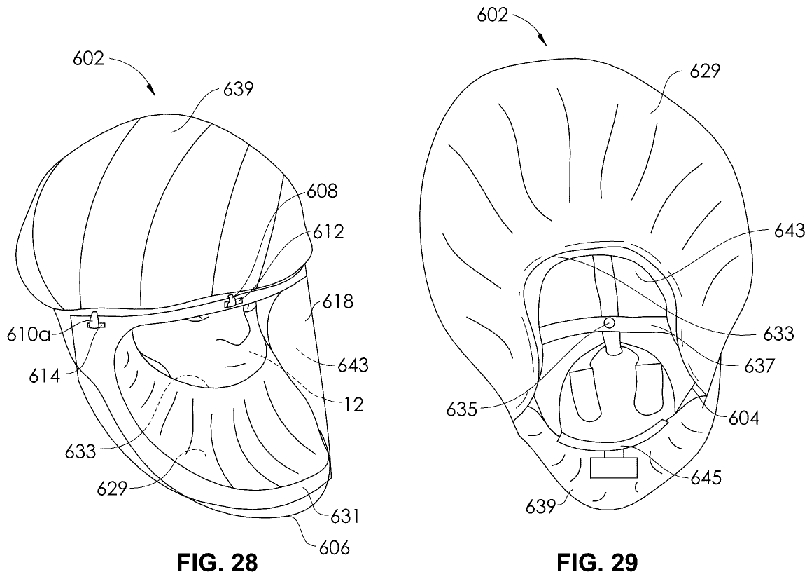

A personal environmental protection system 602 is illustrated in FIGS. 24-27 and comprises a support portion 604 (e.g., head support) and a chin bar 606. The support portion 604 carries a center hook 608 and two side hooks 610a, 610b. The hooks 608, 610a, 610b are configured to engage holes 612, 614, 616 which extend through an upper extremity 627 of a facial shield 618, illustrated in FIG. 27. The facial shield 618 can comprise a clear sheet of the material previously described herein. The facial shield 618, however, further comprises a cuff 629, which may comprise a woven protective barrier. The cuff 629 is bonded to the facial shield by a seam 631 running substantially vertically along the right side 611, substantially horizontally along the lower extremity 625, and substantially vertically along the left side 613 of the facial shield 618. The cuff 629 includes an elastic inner perimeter 633 configured to fit snugly around a user's neck or lower head, to create a protected internal space 643 (FIG. 28). Turning to FIG. 25, the center hook 608 has a base 620 configured to couple to an outer surface 622 of the support portion 604, and a hook portion 624 having a maximum width W₄. The hook portion 624 may have a J-shape or an L-shape, or any other shape that may allow some level of hooking to occur. Each of the side hooks 610a, 610b have a base 626 configured to couple to the outer surface 622 of the support portion 604, and a hook portion 628 having a maximum width W₅. Each hole 614, 616 has a width W₆ that is greater than the maximum width W₅, and the hole 612 has a width W₇ that is greater than the maximum width W₄, thus allowing each hole 612, 614, 616 to fit completely over each hook 608, 610a, 610b. In some embodiments, the width W₆ may be configured to be at least 10% greater than the width W₅, or at least 25% greater than the width W₅. In some embodiments, the width W₇ may be configured to be at least 25% greater than the width W₄, or at least 50% greater than the width W₄, or at least 75% greater than the width W₄, or at least 100% greater than the width W₄. In some embodiments, the width W₆ may be configured to be between about 25% and about 100% greater than the width W₅. In some embodiments, the width W₇ may be configured to be between about 25% and about 100% greater than the width W₄. In some embodiments, the hole 616 has a first lateral edge 617 and a second lateral edge 619. Hole 614 may be described in the same manner. In some embodiments the first and second lateral edges 617, 619 can be simultaneously placed outside of the upper hook portion maximum width (width W₅) of an upper hook portion 640 of the side hook 610b (or side hook 610a) while the hole 612 remains engaged on the center hook 608, but only when the facial shield 618 is in a particular lateral position in relation to the center hook 608. This relation may be adjusted via sliding adjustment.

The hook portion 624 of the center hook 608 has a maximum thickness t3, and the hook portion 628 of the side hooks 610 has a maximum thickness t4. Hole 612 has a gap g₃ that is at least slightly greater than the maximum thickness t3, thus allowing the hole 612 to fit completely over center hook 608. Holes 614, 616 each have a gap g₄ that is at least slightly greater than the maximum thickness t4, thus allowing each hole 614, 616 to fit completely over each side hook 610a, 610b. Center hook 608 has an overhang 630, and a recess 632, between the overhang 630 and the base 626. To attach the facial shield 618 to the support portion 604, hole 612 is first inserted over the end 634 of the overhang 630 and the facial shield 618 is pulled down over the hook portion 624, toward the base 620 so that a border portion 636 of the facial shield 618, that is peripheral to hole 612, fits completely within the recess 632, with the facial shield 618 extending substantially vertically downward. The hole 612 is now locked over the base 620 of the center hook 608 in a somewhat similar manner, though not identical, to the way the second section 503 of the hole 512 is locked over the center hook 508 in FIG. 11. However, the locking is caused only by tension, with no sliding required. In some embodiments, a bottom portion 641 of the hole 612 can be configured to deform or flex, and snap over a bottom edge 649 of the center hook 608, similar to the snapping of the lower edge 548 over the bottom face 550 in FIGS. 13-14.

The facial shield 618 is then moved to the left so that the hole 612 slides to the far left on the hook portion 624 of the center hook 608. The hole 612 remains locked to the center hook 608 while being slid. Next, the hole 614 is inserted over the end 638 of an upper hook portion 640 of the hook portion 628 of the side hook 610a, and the right side 611 of the facial shield 618 is pulled down over the upper hook portion 640 toward the base 626. An upper edge 644 of the hole 614 is forced against an upper ledge 646 of the base 626, and the right side 611 of the facial shield 618 is forced further downward, slightly opening the gap g₄, such that a lower edge 648 of the hole 614 clears a bottom face 650 of a lower hook portion 642 of the hook portion 628 of the side hook 610a. When the hole 614 is released, and loses its slight deformation, the lower edge 648 of the hole 614 is snapped into its locked, rest position, above the bottom face 650, but below a lower ledge 652 of the base 626. The hole 614 is thus locked both in relation to the upper hook portion 640 and the lower hook portion 642. The facial shield 618 thus extends substantially vertically downward at its right side 611, as well as at its center. If needed, the facial shield 618 can now be moved slightly to the left, with the hole 614 sliding (in a locked position) along the side hook 610a and with the hole 612 sliding (in a locked position) along the center hook 608. The snapping process of the hole 614 and the side hook 610a can now be repeated with the hole 616 and the side hook 610b. The facial shield 618 is now securely in place on the support portion 604, with a few quick and simple snap and slide actions, as shown in FIG. 28. Removal of the facial shield 618 can be performed by substantially reversing the actions and steps. In other embodiments, any one or more of the holes 612, 614, 616 may be vertically elongated instead of horizontally elongated. One or more of the holes 612, 614, 616 may even be diagonally elongated.

The personal protection system 240 is illustrated in its assembled configuration in FIGS. 28 and 29. The cuff 629 extends from the seam 631 and terminates in the elastic inner perimeter 633, forming an arc-shaped elastic window. An elastic band 637 is coupled to the cuff 629 (e.g., by sewing) and has a central snap 635 configured to secure the cuff 629 to the support portion 604 via a cooperative snap (adjacent, but not shown) held thereon. The elastic inner perimeter 633 is configured to fit snugly around a user's neck or lower head, to create the protected internal space 643. In some cases, the elastic inner perimeter 633 can be worn on the user's ears. In other cases, the elastic perimeter can be worn below the user's ears. In other cases, the elastic inner perimeter 633 can be pulled completely above or substantially above the user's ears. This particular positioning above the ears is useful, for example, if the user needs to use a stethoscope, which requires access to both ears and extends below the ears. The positioning may also be useful if the user is using one or more earphones (e.g., over ear or earbuds), so that an assistant may remove one of the earphones at a certain time in a procedure. Alternatively, if the user is using one earphone only, the user may choose to have the elastic inner perimeter 633 pulled above one ear and below the other ear. In some embodiments, a woven cloth covering 639 may be carried on and over the support portion 604. In other embodiments, the cuff 629 may be integral with the woven cloth covering 639, such that the window formed by the elastic inner perimeter 633 is first placed over the head of the user, and subsequently the woven cloth covering 639 is attached to the top of the support portion 604 (e.g., stretched over it), and the facial shield 618 is attached to the support portion 604, as described. An adjustable strap 645 may be used to create the appropriate fit around the user's head. The combination of the cuff 629 into the facial shield 618 allows disposability of portions of the personal environmental protection system 602 (facial shield 618, cuff 629, woven cloth covering 639) that may be more difficult to clean or maintain, while preserving reusability of the support portion 604, which may be more easily cleaned and reused. Any of the facial shields or cuffs described herein may be configured from biodegradable materials. Any of the materials described herein may be configured from reusable materials. In other embodiments, the cuff 629 may be incorporated with the facial shield 518 of FIG. 6. In alternative embodiments, any of the holes 512, 514, 516, 612, 614, 616 may be replaced by a hole, a notch, or a groove within a separate standoff or bracket that is attached to the facial shield 518, 618, or in a thickened portion of the facial shield 518, 618 that acts like a standoff or bracket. In other alternative embodiments, one or more of the hole/hook relationships may be replaced or augmented by a magnet/ferrous material relationship, or even a magnet/magnet relationship. For example, the facial shield 518, 618 carries one or more steel or 400 series stainless steel buttons or plates that is/are configured to releasably attached to one or more magnet carried by the outer surface 522, 622.

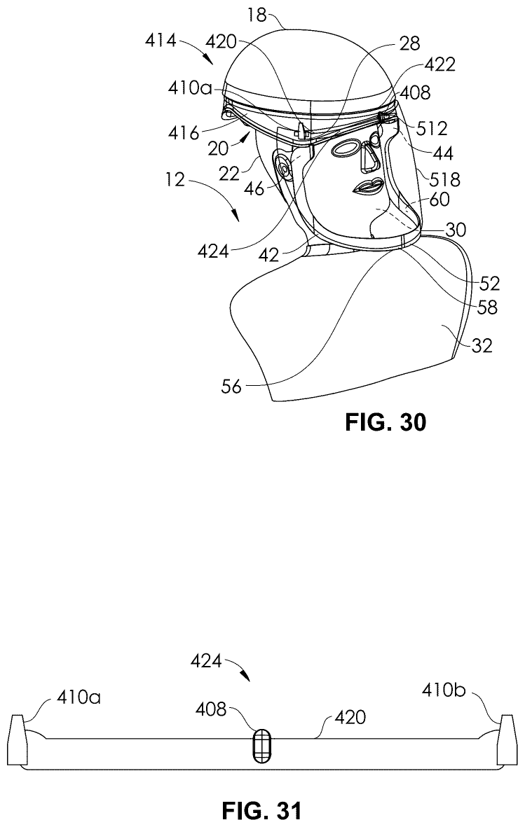

FIG. 30 illustrates a user 12 wearing a personal environmental protection system 414 that is similar to the personal environmental protection system 14 of FIG. 1. However, as illustrated in FIG. 31, a center hook 408 and two side hooks 410a, 410b are carried as a hook assembly 424. The center hook 408 and two side hooks 410a, 410b are carried are attached to a flexible strip 420. A back surface of the flexible strip 420 is attached to an outer surface 422 of a head support 416. By first constructing the hook assembly 424, a precision distance between each hook 408, 410a, 410b can be achieved. The manufacture of the head support 416 can also be simplified, because it does not have to have contours (slits and/or projections) to accommodate each hook 408, 410a, 410b. The flexible strip 420 may have an adhesive strip carried on its back surface. A user can remove a protective cover from the adhesive strip and then attached the flexible strip to the outer surface 422 of the head support 416 via the adhesive strip. The hook assembly can be centered and oriented on the head support 416 via a mark in the center of the head support 416 that corresponds to the location for the center hook 408. Because the head support 416 does not have to have the additional features to accommodate each hook, the head support 416 is more readily cleanable and, if appropriate, sterilizable. The hook assembly 424 can be disposable, or can be reusable, but may be configured for fewer reuses than the head support 416. In alternative embodiments, the backing of the flexible strip 420 may comprise a hooks and loops structure corresponding to a hooks and loop structure carried on the outer surface 422 of the head support 416.

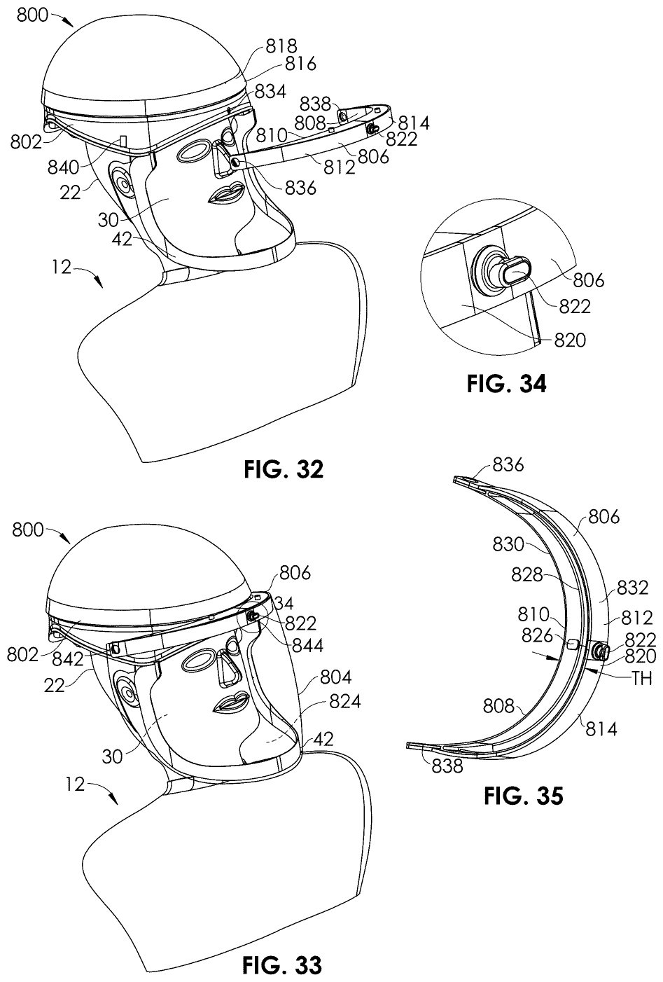

FIGS. 32-35 illustrate an alternative embodiment of a personal protection system 800 placed on the head 22 of a user 12. The personal protection system 800 is similar to other embodiments of the personal protection systems previously described herein, as they include a head support 802 and a facial shield 804. The system 800 may also comprise a chin bar 42. An extender 806 is configured to be directly coupled to the head support 802, as shown in FIGS. 32 and 33. The extender 806 comprises an interior side 808 having a concavity 810, and an exterior side 812 having a first convexity 814. The concavity 810 is configured to closely match a second convexity 816 on an anterior surface 818 of the head support 802. A central hook 822 is carried on the exterior side 812 of the extender 806 at a central portion 820 (FIG. 34) and extends outwardly from the exterior side 816. The extender 806 has a thickness TH (FIG. 35) at the central portion 820. The thickness TH serves to place the central hook 822 further away from the head support 802 to thus create a larger amount of space 824 behind the facial shield 804 (when attached). The increased space may include an increased distance from the face 30 of the user 12 and/or an increased volume surrounding the face 30 of the user 12 (behind the facial shield 804). Thus, additional room/space is created for other components, such as a communication system, or a lighting system, as described further herein. This additional space may also accommodate larger facial features (nose, moustache, beard etc.) or accessories (eyeglasses, loupe, etc.). Furthermore, the additional space may work in a capacitive sense, to allow for better control or flow of air or oxygen supplied internally, to avoid fogging of the facial shield 804 (e.g., by the increased distance from the nose and mouth to the facial shield 804), or to avoid undesired increase of internal carbon dioxide levels.

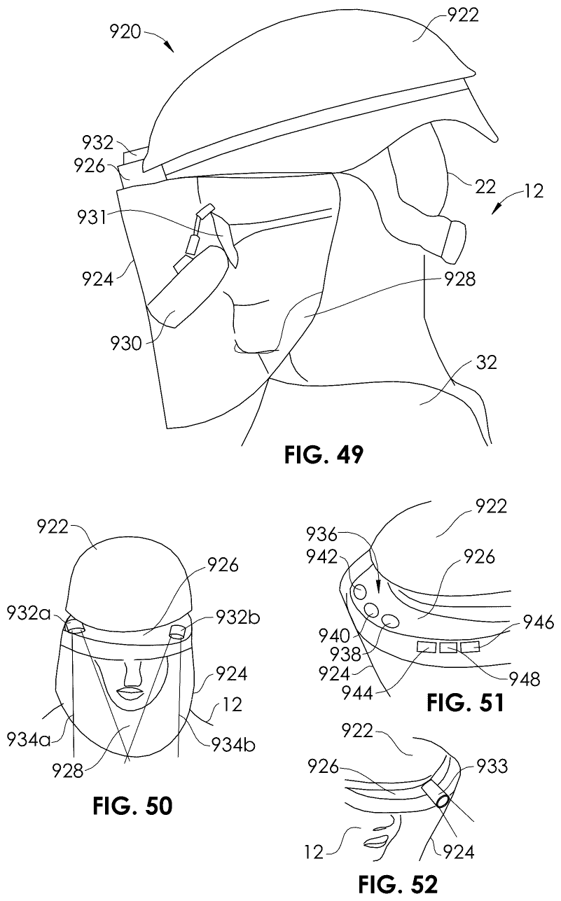

The extender 806 includes a hole 826 on its interior side 808, adjacent a cut-away space 828. The cut-away space 828 serves to make the extender lighter in weight, and may aid fabrication, such as injection molding, or lower cost. The hole 826 passes through an interior wall 830, which is opposite an exterior wall 832, on the other side of the space 828. The hole 826 is configured to be engaged to a hook 834 (FIG. 32) on the anterior surface 818 of the head support 802. Side holes 836, 838 are configured to be engaged onto side hooks 840 carried on the head support 802. The side hooks 840 may be configured to not only allow the extender 806 so attach to the head support (via the side holes 836, 838), but may also be configured to allow the engagement of end holes 842 of the facial shield 804 (over the extender 806). Any of the hook configurations and/or mechanisms described herein may be utilized for the center hook 822, the hook 834, or the side hooks 840. In some embodiments, the center hook 822 may be rotatable to allow it to lock to a center hole 844 of the facial shield 804. For example, the hook 834 may be rotated 90° after placement of the facial shield 804, to lock the facial shield 804 in place. After use, the extender 806 may be reusable (cleanable, resterilizable), or may be removable and disposable with the facial shield 804. The extender 806 serves to make the head support 802 adaptable to a variety of different setups. The extender 806 may be configured to carry lights (e.g., LEDs) and/or cameras, as disclosed in more detail in the embodiments of FIGS. 49-52.

Certain embodiments are described in the following clauses.
Clause 1—A protective headgear system includes: a support configured to engage the head of a user; a facial shield including a polymeric sheet having an upper portion configured to be coupled to the support and a lower portion having a lower extremity, wherein the upper portion includes a first hole, a second hole, and a third hole, each one of the first, second, and third holes substantially spaced from the other two of the first, second, and third holes, and wherein the first and second holes are each substantially elongated; a first hook carried on the support, wherein the first hole is configured to be engaged with first hook; a second hook carried on the support, wherein the second hole is configured to be engaged with second hook; a third hook carried on the support, wherein the third hole is configured to be engaged with third hook; wherein the first hook includes a hook portion having a maximum hook width, the first hook located on a perimeter of the support between the second and third hooks; and wherein the first hole has a first hole gap and a first hole width, the first hole width of the first hole at least about 25% greater than the maximum hook width of the hook portion of the first hook.
Clause 2—The system of clause 1, wherein the first hole extends substantially horizontally.
Clause 3—The system of clause 2, wherein the second hole extends substantially horizontally.
Clause 4—The system of clause 3, wherein the third hole is substantially elongated and extends substantially horizontally.
Clause 5—The system of clause 1, wherein the second hook includes a base coupled to the support, an upper hook portion extending substantially vertically upward from the base, and a lower hook portion extending substantially vertically downward from the base.
Clause 6—The system of clause 5, wherein the third hook includes a base coupled to the support, an upper hook portion extending substantially upward from the base, and a lower hook portion extending substantially downward from the base.
Clause 7—The system of clause 5, wherein the upper hook portion of the second hook has an upper hook portion maximum thickness and an upper hook portion maximum width, and wherein the second hole has a second hole gap and a second hole width, the second hole width of the second hole at least about 25% greater than the maximum width of the upper hook portion of the second hook.
Clause 8—The system of clause 7, wherein the second hole width of the second hole is between about 25% and about 100% greater than the maximum width of the upper hook portion of the second hook.
Clause 9—The system of clause 7, wherein the upper hook portion maximum thickness of the upper hook portion of the second hook is less than the second hole gap of second hole.
Clause 10—The system of clause 7, wherein the second hole has a first lateral edge and a second lateral edge, and wherein the first and second lateral edges of the second hole can be simultaneously placed outside of the upper hook portion maximum width of the upper hook portion of the second hook while the first hole remains engaged on the first hook.
Clause 11—The system of clause 5, wherein the base of the second hook includes a hole-carrying portion adjacent to one or both of the upper hook portion and lower hook portion, the hole-carrying portion having a having a vertical thickness less than the second hole gap of the second hole, the hole-carrying portion further having a horizontal thickness less than the second hole width of the second hole.
Clause 12—The system of clause 11, wherein the lower hook portion of the second hook has a vertical extension length from a lower portion of the base of the second hook, wherein the sum of (a) the vertical extension length of the lower hook portion of the second hook and (b) the vertical thickness of the hole-carrying portion of the base of the second hook is greater than (c) the second hole gap of the second hole.
Clause 13—The system of clause 1, wherein the first hook includes a J-shape.
Clause 14—The system of clause 1, wherein the first hook includes an L-shape.
Clause 15—The system of clause 1, wherein the first hole width of the first hole is between about 25% and about 100% greater than the maximum width of the hook portion of the first hook.
Clause 16—The system of clause 1, wherein the hook portion of the first hook has a maximum thickness that is less than the first hole gap of first hole.
Clause 17—The system of clause 1, wherein each of the second hook and third hook is located on the perimeter of the support.
Clause 18—The system of clause 1, wherein the facial shield includes polyester.
Clause 19—The system of clause 18, wherein the facial shield includes PETG.
Clause 20—The system of clause 18, wherein the facial shield includes PET.
Clause 21—The system of clause 1, wherein the lower portion of the facial shield does not include holes.
Clause 22—The system of clause 1, wherein the facial shield includes a high clarity polymer.
Clause 23—The system of clause 1, wherein the facial shield includes a die-cut sheet.
Clause 24—The system of clause 23, wherein the first, second, and third holes are die-cut.
Clause 25—The system of clause 23, wherein the first, second, and third holes are punch-formed.
Clause 26—The system of clause 1, wherein the facial shield has a thickness of between about 0.010 inch and about 0.020 inch.
Clause 27—The system of clause 1, further including a filter carried by the support and capable of filtering air.
Clause 28—The system of clause 27, further including a fan configured to more air through the filter and into a space between the user and the facial shield when the support is engaged on the head of the user.
Clause 29—The system of clause 1, wherein the facial shield is biodegradable.
Clause 30—The system of clause 1, wherein the facial shield is reusable.
Clause 31—The system of clause 1, further including a cuff coupled to the lower portion of the facial shield.
Clause 32—The system of clause 31, wherein the cuff includes a first portion coupled to the lower portion of the facial shield and a second portion, opposite the first portion, the second portion configured to engage the neck of the user.
Clause 33—A protective headgear system includes: a support configured to engage the head of a user; a facial shield including a polymeric sheet having an upper portion configured to be coupled to the support and a lower portion having a lower extremity, wherein the upper portion includes a first hole, a second hole, and a third hole, each one of the first, second, and third holes laterally spaced from the other two of the first, second, and third holes; a first hook carried on the support, wherein the first hole is configured to be engaged with first hook; a second hook carried on the support, wherein the second hole is configured to be engaged with second hook; a third hook carried on the support, wherein the third hole is configured to be engaged with third hook, wherein the first hook is located on a perimeter of the support between the second hook and the third hook; wherein the first hook includes a hook portion having a maximum hook width, and wherein the first hole has a lateral width greater than the maximum hook width of the hook portion of the first hook; and wherein the facial shield has a first position in relation to the support such that the first hole can be placed over the hook portion of the first hook and the second hole is not oriented to be coupled to the second hook, and wherein the first hole is laterally slidable in relation to the first hook such that the facial shield is locked to the first hook via the first hole and such that the facial shield has a second position in relation to the support, wherein the second hole is oriented to be coupled to the second hook.
Clause 34—The system of clause 33, wherein when the facial shield is in the second position in relation to the support, the third hole can be placed in position to be coupled to the third hook.
Clause 35—The system of clause 33, wherein the second hook includes a base coupled to the support, an upper hook portion extending substantially vertically upward from the base, and a lower hook portion extending substantially vertically downward from the base.
Clause 36—The system of clause 35, wherein the third hook includes a base coupled to the support, an upper hook portion extending substantially upward from the base, and a lower hook portion extending substantially downward from the base.
Clause 37—The system of clause 35, wherein the second hole has a second hole gap and a second hole width, and wherein the base of the second hook includes a hole-carrying portion adjacent to one or both of the upper hook portion and lower hook portion, the hole-carrying portion having a having a vertical thickness less than the second hole gap of the second hole, the hole-carrying portion further having a horizontal thickness less than the second hole width of the second hole.
Clause 38—The system of clause 37, wherein the lower hook portion of the second hook has a vertical extension length from a lower portion of the base of the second hook, wherein the sum of (a) the vertical extension length of the lower hook portion of the second hook and (b) the vertical thickness of the hole-carrying portion of the base of the second hook is greater than (c) the second hole gap of the second hole.
Clause 39—The system of clause 35, wherein the second hole is configured to be slid over the upper hook portion of the second hook and snapped over the lower hook portion of the second hook, thereby locking the facial shield to the second hook via the second hole.
Clause 40—The system of clause 36, wherein the third hole is configured to be slid over the upper hook portion of the third hook and snapped over the lower hook portion of the third hook, thereby locking the facial shield to the third hook via the third hole.
Clause 41—The system of clause 33, wherein the first hook includes a base coupled to the support, an upper hook portion extending substantially upward from the base, and a lower hook portion extending substantially downward from the base.
Clause 42—The system of clause 41, wherein the first hook has a maximum vertical dimension and a vertical base thickness, and wherein the first hole has a first section having a first hole gap and a second section laterally adjacent the first section and having a second hole gap, wherein the first hole gap of the first hole is greater than or equal to the maximum vertical dimension of the first hook, and wherein the second hole gap of the first hole is greater than the vertical base thickness and is less than the maximum vertical dimension.
Clause 43—The system of clause 33, wherein the distance along the perimeter between the second hook and the third hook is between about twelve inches and about fourteen inches.
Clause 44—The system of clause 33, wherein the distance between a horizontal center of the second hole and a horizontal center of the third hole is between about twelve inches and about fourteen inches.
Clause 45—The facial shield of clause 33, further including a cuff coupled to the lower portion of the facial shield.
Clause 46—The facial shield of clause 45, wherein the cuff includes a first portion coupled to the lower portion of the facial shield and a second portion, opposite the first portion, the second portion configured to engage the neck of the user.
Clause 47—A facial shield for a protective headgear system including a support configured to engage the head of a user and first, second, and third hooks carried on the support, wherein the first hook is located on a perimeter of the support between the second hook and the third hook, and wherein the first hook includes a hook portion having a maximum vertical dimension and a base coupled to the support and having a vertical base thickness, the facial shield including: a polymeric sheet having an upper portion configured to be coupled to the support and a lower portion having a lower extremity, wherein the upper portion includes a first hole, a second hole, and a third hole, each one of the first, second, and third holes laterally spaced from the other two of the first, second, and third holes; and the first hole including a first section having a first hole gap and a second section laterally adjacent the first section and having a second hole gap, wherein the first hole gap of the first hole is greater than or equal to the maximum vertical dimension of the first hook, and wherein the second hole gap of the first hole is greater than the vertical base thickness of the base of the first hook and is less than the maximum vertical dimension of the first hook.
Clause 48—The facial shield of clause 47, wherein the second hole gap is between about 0.225 inch and about 0.68 inch.
Clause 49—The facial shield of clause 48, wherein the second hole gap is between about 0.30 inch and about 0.45 inch.
Clause 50—The facial shield of clause 47, wherein the first hole gap is greater than or equal to 0.68 inch.
Clause 51—The facial shield of clause 47, wherein the second hole is horizontally elongated.
Clause 52—The facial shield of clause 51, wherein the third hole is horizontally elongated.
Clause 53—The facial shield of clause 47, further including a cuff coupled to the lower portion of the polymeric sheet.
Clause 54—The facial shield of clause 53, wherein the cuff includes a first portion coupled to the lower portion of the polymeric sheet and a second portion, opposite the first portion, the second portion configured to engage the neck of the user.
Clause 55—The facial shield of clause 47, wherein the second hole is snappably engageable with the second hook.
Clause 56—The facial shield of clause 55, wherein the third hole is snappably engageable with the third hook.
Clause 57—The facial shield of clause 47, wherein the facial shield has a first position in relation to the support such that the first hole can be placed over the hook portion of the first hook and the second hole is not oriented to be coupled to the second hook, and wherein the first hole is laterally slidable in relation to the first hook such that the facial shield is locked to the first hook via the first hole and such that the facial shield has a second position in relation to the support, wherein the second hole is oriented to be coupled to the second hook.
Clause 58—The system of clause 47, wherein the polymeric sheet has a thickness of between about 0.010 inch and about 0.020 inch.
Clause 59—The system of clause 47, wherein the facial shield is configured to be coupled to the support without hand contact to the support.
Clause 60—The system of clause 47, wherein the facial shield is configured to be coupled to the support without glove or hand contact to the support.
Clause 61—A method for donning a protective headgear system including a support configured to engage the head of a user and first, second, and third hooks carried on the support, wherein the first hook is located on a perimeter of the support between the second hook and the third hook, and wherein the first hook includes a hook portion having a maximum vertical dimension and a base coupled to the support and having a vertical base thickness, the method including: providing a polymeric sheet having an upper portion configured to be coupled to the support and a lower portion having a lower extremity, wherein the upper portion includes a first hole, a second hole, and a third hole, each one of the first, second, and third holes laterally spaced from the other two of the first, second, and third holes, wherein the first hole includes a first section having a first hole gap and a second section laterally adjacent the first section and having a second hole gap, wherein the first hole gap of the first hole is greater than or equal to the maximum vertical dimension of the first hook, and wherein the second hole gap of the first hole is greater than the vertical base thickness of the base of the first hook and is less than the maximum vertical dimension of the first hook; placing the first hole over the first hook; sliding the polymeric sheet in relation to the support to lock the first hole to the first hook; attaching the second hook to the second hole; and attaching the third hook to the third hole.
Clause 62—The method of clause 61, wherein the attachment of the second hook to the second hole includes snapping the second hole over the second hook.
Clause 63—The method of clause 62, wherein the attachment of the third hook to the third hole includes snapping the third hole over the third hook.

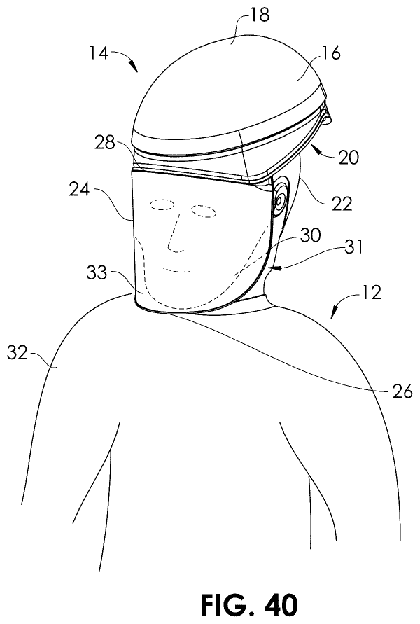

FIG. 40 illustrates a user 12 wearing a personal environmental protection system 14. The user 12 may be a surgeon, physician, or another medical personnel participating in a surgery or other medical procedure on a patient that has an ailment that is contagious in an otherwise standard, unprotected environment. The personal environmental protection system 14 comprises a head support 16 having a top portion 18 and a concave underside 20 configured to engage with the head 22 of the user 12. The head support 16 may comprise a helmet or other type of headgear that is securely and comfortably maintainable on the head 22 of the user 12 during an entire medical procedure, to avoid the need for constant removal, adjustment or replacement. A facial shield 24 (or lens) comprising a substantially optically clear material includes a lower extremity 26 configured to extend below the face 30 of the user 12, and an upper portion 28 configured to be coupled to the head support 16. The user 12 is able to clearly view through the facial shield 24 while the head support 16 is engaged with the user's 12 head 22. Thus, upon movement of the head 22 of the user 12, the head support 16 maintains the facial shield 24 in front of the face 30 of the user 12. In some embodiments, a gown 32 or toga may be used by the user 12 to protect some, most, or all of the user's 12 body below the head and neck, or at least to cover the upper torso of the user 12. In some embodiments, the gown 32 may couple directly to the facial shield 24, the head support 16, or both the facial shield 24 and the head support 16. The personal environmental protection system 14 may be configured to substantially control the user's 12 breathing environment via air filtration, inflow, and/or outflow, and may include some or all features of any of the embodiments described in U.S. Pat. No. 8,302,599 to Green issued Nov. 6, 2012, and entitled “Protective Headgear System with Filter Protector.” The personal environmental protection system 14 may also or alternatively include some or all features of any of the embodiments described in U.S. Pat. No. 8,453,262 to Green issued Jun. 4, 2013, and entitled “Personal Environmental Protection Apparatus.”

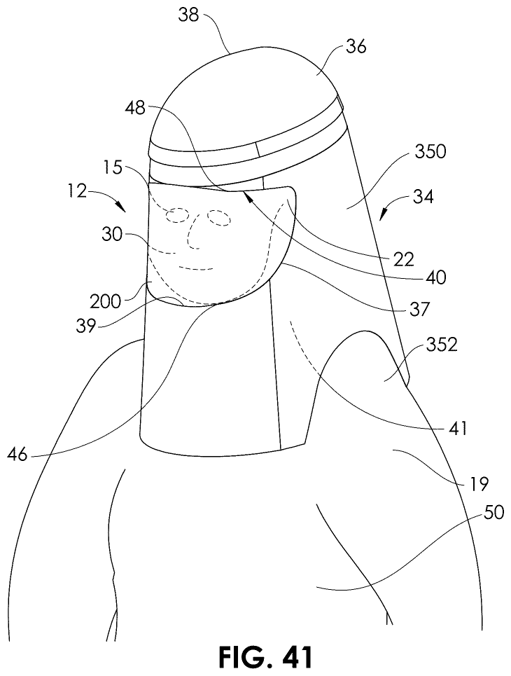

FIG. 41 illustrates a user 12 wearing an alternative personal environmental protection system 34 comprising a hood 350. The hood 350 includes a support portion 36, configured to engage with the head 22 of the user 12. The support portion 36 is a direct extension of the hood 350 and includes a top portion 38 and a concave underside 40 for reversible coupling to the head 22 of the user 12. The hood 350 comprises a facial shield 200 comprising a substantially optically clear material. The user 12 is able to clearly view through the facial shield 200 while the support portion 36 is engaged with the user's 12 head 22. The facial shield 200 has an outer periphery 37 that is sealably secured to a window 39 in the hood 350, to provide a protected and isolated interior space 41. The facial shield 200 includes a lower extremity 46 configured to extend at least below the eyes 15 of the user 12. In the embodiment illustrated in FIG. 41, the lower extremity 46 of the facial shield 200 extends substantially below the eyes 15 of the user 12 and extends to a region near the chin (not shown) of the user 12. The facial shield 200 further comprises an upper portion 48 configured to be coupled to the support portion 36 (e.g., by the sealed securement, such as adhesives, epoxies, or heat sealing). Thus, upon movement of the head 22 of the user 12, the support portion 36 maintains the facial shield 200 in front of the face 30 of the user 12. The hood 350 also includes a left cutout 352 and a right cutout (not shown) that are configured to form and fit over a gown 50 at the shoulders 19 of the user 12. In some embodiments, the hood 350 may extend like a shroud to substantially cover most or all of the body of the user 12.

The facial shield 24, 200 commonly comprises a substantially rigid, high clarity polymer such as polyethylene terephthalate glycol (PETG), polyethylene terephthalate (PET), or other polyesters or polyester copolymers, or polycarbonate, such that it can provide a relatively thin but tough barrier that does not significantly impede the vision of the user. In some embodiments, the facial shield 24, 200 has a concave shape toward the user 12 and a convex shape away from the user 12. In FIG. 40, a concavity 31 and a convexity 33 are indicated. The material, thickness, and shape of the facial shield 24, 200, either as independent factors or as cooperative factors in any combination, can cause the facial shield 24, 200 to be a sound reflector, reflecting a noticeable portion of generated sound. In some cases, the user 12 may give commands that are not understood by other medical personnel or by an awake patient, because much of the sound of the user's 12 voice is reflected back inside the interior space 41. In other cases, a user 12 may not hear a patient or other medical personnel well, because of the same reflective characteristics of the facial shield 24, 200, reflecting much of the sound away from the user 12. Other factors may exacerbate the inability for the user 12 to hear others. Awake patients may be sick, or may have been given sedatives or other medications, thus slowing or slurring their speech. In other cases, the patients may be in a particular position for the purposes of the procedure that is not conducive to normal speaking mechanics. All of these reasons may make it difficult for the patient to produce sufficient sound energy when speaking that conducts (carries) through the facial shield 24, 200 and to the ears of the user 12, as well as clarity of diction. Thus, a physician may often have trouble understanding a patient. Other medical personnel in the room may moving around while speaking, or may be facing away from the user 12. Thus, this may also make it difficult for these personnel to produce sufficient sound energy when speaking that conducts (carries) through the facial shield 24, 200 and to the ears of the user 12.

Gown 32, 50 may comprise a number of different materials and configurations. Materials for the gown 32, 50 may include a tri-laminate comprising a film held between two layers of non-woven plastic fabric. Bi-laminate materials are also possible, such as a material comprising a film layer and a non-woven plastic fabric. In some embodiments, the non-woven layer or layers may comprise a cellulose. In some embodiments, the non-woven layer may comprise spun materials such as spunbonded high density polyethylene (e.g., Tyvek®, a trademark of DuPont de Nemours, Inc.). In one embodiment a spunbond meltblown spunbond, commonly known as SMS, may be used, and comprises a tri-laminate non-woven fabric comprising a top layer of spunbond polypropylene, a middle layer of meltblown polypropylene and a bottom layer of spunbond polypropylene. In other embodiments, one or more of the non-woven layers may be replaced by a woven layer.

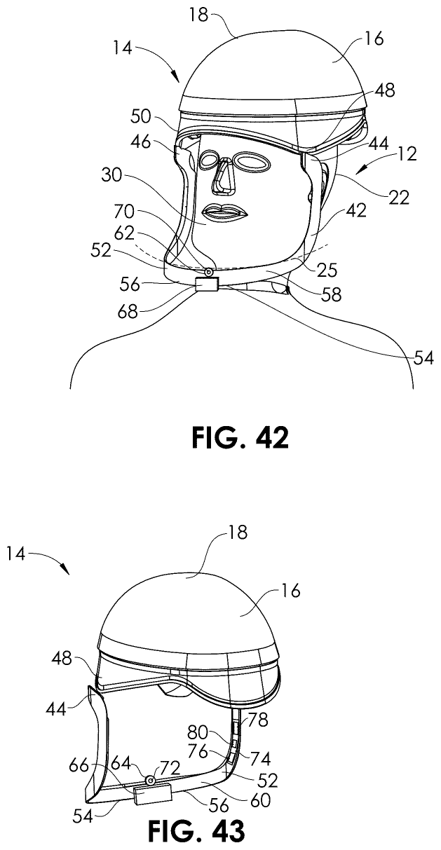

FIGS. 42 and 43 illustrate the personal environmental protection system 14 of FIG. 40 in place on a user 12 with the facial shield 24 and gown 32 removed from view for better visualization of internal details. The lower extremity 26 of the facial shield 24 generally extends to line 25. A chin bar 42 has a first end 44 connected to a first portion 48 of the head support 16 and a second end 46 connected to a second portion 50 the head support 16. The chin bar 42 may comprise a rigid polymeric material such as polyamide, for example nylon 6, or polycarbonate, allowing one of the dimensions of the chin bar 42 to be relatively thin, such that it has a generally rectangular cross-section. The chin bar 42 may alternatively comprise high-density polyethylene or polypropylene, though the thin dimension would likely be thickened somewhat to maintain sufficient stiffness and resistance to extreme bending. The chin bar 42 extends between the first end 44 and the second end 46 in a generally U-shape 52 having a lower apex 54. The lower apex 54 is located at the center of a lower portion 56 of the chin bar 42 having an externally-facing surface 58 and an internally-facing surface 60. The lower portion 56 generally extends laterally in relation to the head 22 of the user 12.

The personal environmental protection system 14 is configured to provide a two-way communication system that solves the sound problems caused by the facial shield 24. The chin bar 42 serves as a controlled mounting structure for microphones 62, 64 and loudspeakers 66, 68 that are configured for providing controlled two-way verbal communication. A first microphone 62 is carried on the externally-facing surface 58 of the chin bar 42 at the lower portion 56, and is configured to sense sound coming from the environment of the operating theater or procedure room outside of the personal environmental protection system 14. The first microphone 62 includes a diaphragm 70, or pickup, or other sensor of sound energy and is preferentially oriented away from the user 12, to thus receive soundwaves in the surrounding area. The first microphone 62 is configured to output a first signal based on the measured sound. A first loudspeaker 66 is carried on the internally-facing surface 60 of the chin bar 42 at the lower portion 56, and is configured to convert the first signal into audio that can be heard by the user 12 within the internal environment of the personal environmental protection system 14. In some embodiments, the first loudspeaker 66 may be replaced by an earphone, either an internal earphone or earbud configured to be inserted into an ear canal of the user 12, or an external earphone configured to be carried on the outer ear of the user 12. In some embodiments, both a loudspeaker and an earphone may be used. The combination of the first microphone 62 (or multiple, arrayed microphones) and first loudspeaker 66, or alternatively one or more microphone 62 and one or more earphone, serves to provide sound information originally generated external to the facial shield 24 to the user 12 whose ears are within the facial shield 24.

A second microphone 64 is carried on the internally-facing surface 60 of the chin bar 42 and is configured to sense sound coming from the voice of the user 12. The second microphone 64 includes a diaphragm 72, or pickup, or other sensor of sound energy and is preferentially oriented toward the mouth or throat of the user 12, to thus receive soundwaves in the local area generated by the user's 12 speech. The second microphone 64 is configured to output a second signal based on the measured sound. A second loudspeaker 68 is carried on the externally-facing surface 58 of the chin bar 42 at the lower portion 56, and is configured to convert the second signal into audio that can be heard by medical personnel or the patient, outside of the internal environment of the personal environmental protection system 14. In some embodiments, one or more of the medical personnel or even the patient may use an earphone, either an internal earphone or earbud configured to be inserted into an ear canal, or an external earphone configured to be carried on the outer ear. In some embodiments, both a loudspeaker and an earphone may be used. The combination of the second microphone 64 (or multiple, arrayed microphones) and second loudspeaker 68, or alternatively one or more microphone 64 and one or more earphone, serves to provide sound information generated by the user 12 within the facial shield 24 to persons external to the facial shield 24. In other embodiments, one or more of the microphones 62, 64 and loudspeakers 66, 68 may be attached to other portions of the chin bar 42, or attached to other items entirely, while maintaining the same general orientation necessary for the functions described herein.

A flex circuit 74 is carried on the chin bar 42, either on the internally-facing surface 60 (as shown) or on the externally-facing surface 58. The flex circuit 74, the first and second microphones 62, 64 and the first and second loudspeakers 66, 68 may each be coupled directly to the chin bar 42 (e.g., by epoxy, fasteners, adhesive, or mechanical snaps), or may be hung from another part of the personal environmental protection system 14 in a manner such that each is adjacent the chin bar 42 at roughly the same locations as shown in FIGS. 42 and 43. When bonded to the chin bar 42 with epoxy or adhesive, acoustic components such as microphones 62, 64 or loudspeakers 66, 68 by be bonded with epoxy or adhesive having a particular acoustic impedance (when set or cured) such that no significant acoustical interference is imparted onto the acoustic component. In some embodiments, acoustic impedance matching is used. In other embodiments, acoustic impedance mismatching is used. The flex circuit 74 may include one or more amplifier 76 configured to amplify the first signal and/or second signal. The flex circuit 74 may also include a processor 78 and a memory 80 configured to digitally record any of the audio information measured, amplified, or generated. The recording can be used as a dictation of the procedure, saving a physician time, and obtaining the critical information when it is freshest in the physician's memory. The user may be able to give voice commands to increase the sound record dB or increase the loudspeaker or earphone volumes.

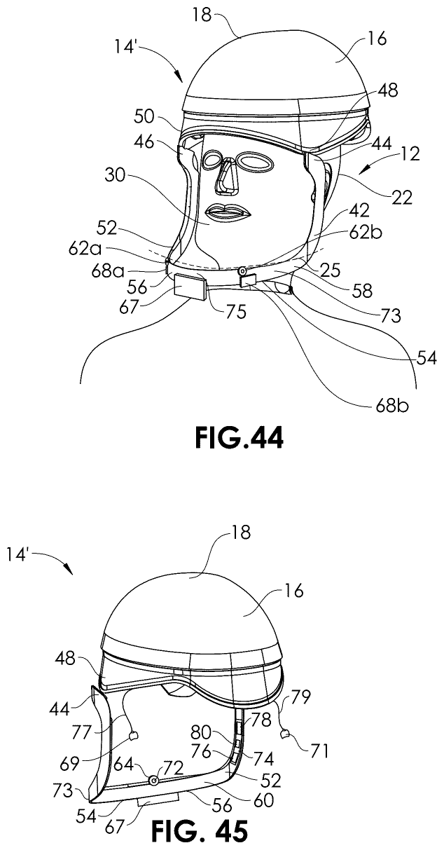

An alternative personal environmental protection system 14′ is illustrated in FIGS. 44 and 45, and has similar elements as the personal environmental protection system 14 of FIGS. 40, 42, and 43. However, a pair of microphones 62a, 62b are carried on two different locations on the externally-facing surface 58, and a pair of loudspeakers 68a, 68b are carried on two different locations on the externally-facing surface 58. Furthermore, the loudspeaker 66 is replaced by a left earphone 69 and a right earphone 71. The earphones 69, 71 are shown having conductive wires 77, 79 to deliver signals to them, respectively, however, in alternative embodiments, the earphones 69, 71 may receive signals wirelessly, for example, by Bluetooth® (Bluetooth SIG, Inc.). The pair of microphones 62a, 62b and pair of loudspeakers 68a, 68b are oriented along two axes that are non-parallel. In other words, the microphone 62a is oriented in a non-parallel manner relative to the microphone 62b, and the loudspeaker 68a is oriented in a non-parallel manner relative to the loudspeaker 68b. Thus, microphone 62a may preferentially sense sound from a first part of a room, while microphone 62b preferentially senses sound from a second, different part of a room. In some embodiments, unidirectional microphones may be utilized. Likewise, loudspeaker 68a preferentially delivers audio to a first part of a room, while loudspeaker 68b preferentially delivers audio to a second part of a room. In some embodiments, each of the microphone 62a, microphone 62b, loudspeaker 68a, and loudspeaker 62b may be oriented along a different, unique central axis, thus creating four non-parallel axes. However, in some cases, there may be parallelism between, for example, the axis of a single microphone and the axis of a single loudspeaker. The creation of the multiple axes is facilitated by a convex curvature 73 of the lower portion 56 of the chin bar 42. Thus, by mounting each of the microphones 62 and loudspeakers 68 at a different location along the chin bar 42, each of the preferential axes can be easily achieved. In some embodiments, each of the microphones 62 and loudspeakers 68 may also be pivotable, in three axes, so that a preferred orientation can be achieved for each. In the personal environmental protection system 14′, a battery 67 is carried on a central location 75 on the chin bar 42 adjacent the externally-facing surface 58. The central location 75 distributes the weight of the battery 67, creating good balance of the personal environmental protection system 14′ on the user 12. This thus aids comfort and ergonomics for the user 12. The microphones 62 and loudspeakers 68 together have a mass of less than about 500 grams, or less than about 200 grams, or less than about 100 grams, or less than about 50 grams. In some embodiments, the earphones 69, 71 may incorporate a noise cancelation system for additional clarity. In other embodiments, the earphones 69, 71, or alternatively internal loudspeakers may also aided by additional baffles or channels to either move circulating air of the system away from the ears of the user 12, or to simply block the area from the circulating air.

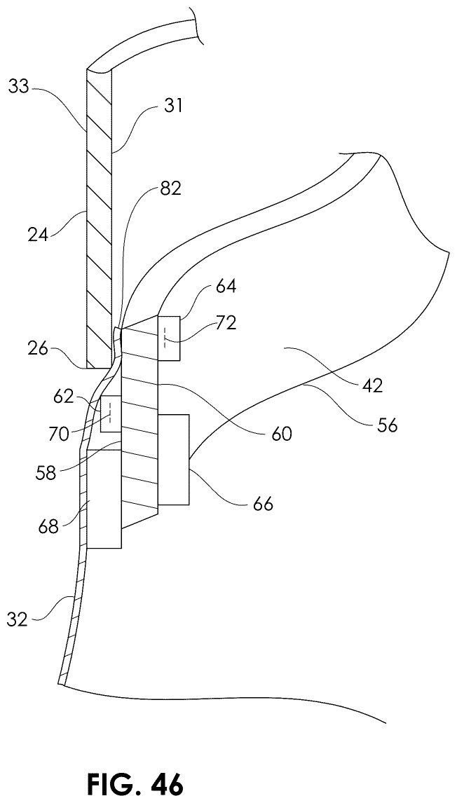

FIG. 46 is a cross-sectional view of the chin bar 42 along an anteroposterior plane in proximity to or comprising the median plane, showing particular mounting locations that allow the first microphone 62 and the second loudspeaker 68 to avoid problems associated with sound reflection by the facial shield 24. The first microphone 62 and the second loudspeaker 68 are each located fully below the lower extremity 26 of the facial shield 24. The upper end 82 of the gown 32 is secured between the facial shield 24 and the chin bar 42. The materials described for fabricating the gown 32 are significantly more transmitting of sound energy than the materials used in the facial shield 24, and thus the location of the first microphone 62 and the second loudspeaker 68 within the gown 32 allows the first microphone 62 and the second loudspeaker 68 to be physically protected (from blood or body fluid splatter or from physical damage), while not significantly affecting their functions.

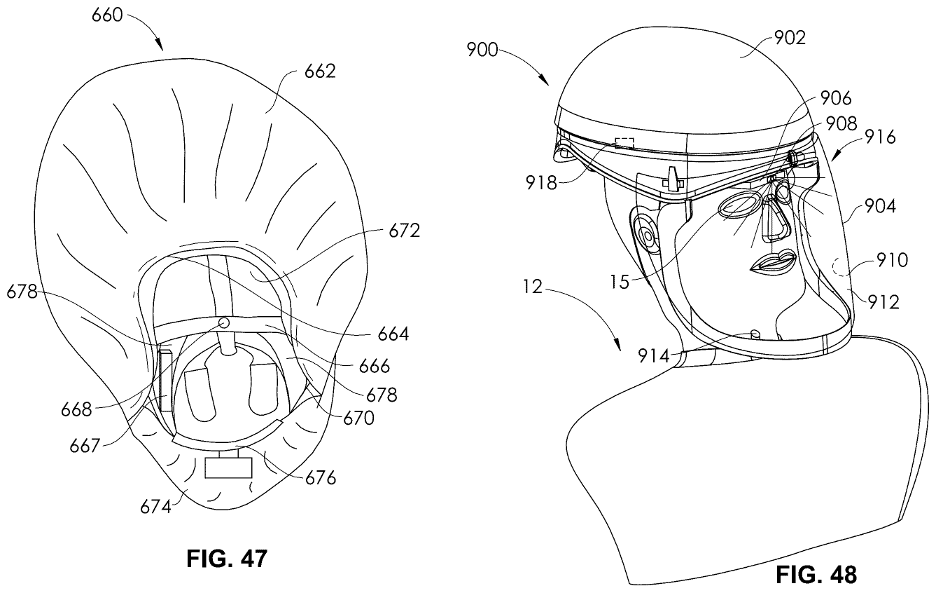

A personal protection system 660 is illustrated in its assembled configuration in FIG. 47. A cuff 662 extends terminates in the elastic inner perimeter 664, forming an arc-shaped elastic window. An elastic band 666 is coupled to the cuff 662 (e.g., by sewing) and has a central snap 668 configured to secure the cuff 662 to the support portion 670 via a cooperative snap (adjacent, but not shown) held thereon. The elastic inner perimeter 664 is configured to fit snugly around a user's neck or lower head, to create the protected internal space 672. In some cases, the elastic inner perimeter 664 can be worn on the user's ears. In other cases, the elastic perimeter can be worn below the user's ears. In other cases, the elastic inner perimeter 664 can be pulled completely above or substantially above the user's ears. This particular positioning above the ears is useful, for example, if the user needs to use a stethoscope, which requires access to both ears and extends below the ears. In some case, a stethoscope having earbuds and utilizing Bluetooth® for wireless auscultation may be utilized. The positioning may also be useful if the user is using one or more earphones (e.g., over ear or earbuds), so that an assistant may remove one of the earphones at a certain time in a procedure. Alternatively, if the user is using one earphone only, the user may choose to have the elastic inner perimeter 664 pulled above one ear and below the other ear. In some embodiments, a woven cloth covering 674 may be carried on and over the support portion 670. In other embodiments, the cuff 662 may be integral with the woven cloth covering 674, such that the window formed by the elastic inner perimeter 664 is first placed over the head of the user, and subsequently the woven cloth covering 674 is attached to the top of the support portion 670 (e.g., stretched over it), and the facial shield (not shown) is attached to the support portion 670. An adjustable strap 676 may be used to create the appropriate fit around the user's head. The combination of the cuff 662 into the facial shield allows disposability of portions of the personal environmental protection system 660 (facial shield, cuff 662, woven cloth covering 674) that may be more difficult to clean or maintain, while preserving reusability of the support portion 670, which may be more easily cleaned and reused. As an alternative to the battery 67 of the embodiment of FIGS. 44-45 being carried on the chin bar 42, a battery 667 is carried within a recess 678, or pocket, within the support portion 670. The support portion 670 can be molded with one or more recess 678 to minimize the overall weight and to increase ease of molding (consistent wall thicknesses, etc.). The placement of the battery 667 within the recess 678 thus takes advantage of this hidden-away location to hide and protect the battery.

FIG. 48 illustrates a personal protection system 900 comprising a head support 902 and a facial shield 904. A video projector 906 is carried by the head support 902 such that the projection lens 908 of the projector 906 points toward the internal surface 910 of the facial shield 904. Thus, the facial shield 904 serves as a projection screen for the user 12. In alternative embodiments, the personal projection system 900 may incorporate the extender 806 of FIGS. 32-35 to create a larger distance between the internal surface 910 of the facial shield 904 and the eyes 15 of the user 12. The projector 906 may comprise any standard palm-size or micro projector, and may be powered by a battery 67, 667 or by an external power source via a power cable. In alternative embodiments, the projector 906 (or an additional projector) may extend outside of the facial shield 904 and may be configured to project onto an external surface 912 of the facial shield 904, for example, to provide visualization of information or even of procedural video to other in the room. In certain embodiments, an audio communication system 914, such as one of those described in the embodiments of FIGS. 42-46, is provided along with the visual communication system 916 (the internal surface 910 and the projector 906), thus giving the user 12 full self-contained communication capabilities. In some embodiments, NIOSH (U.S. National Institute for Occupational Safety and Health) PAPR100 communication performance standards are included.

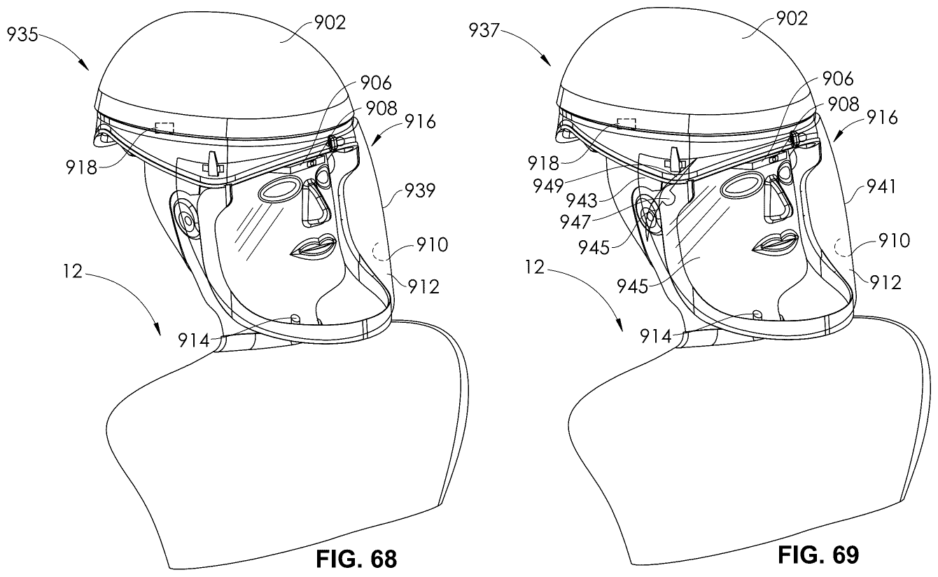

The audio communication system 914 and/or the visual communication system 916 may be configured to provide messages, such as audio messages or text messages to the user 12. The audio communication system 914 may be configured to record and/or transmit audio messages or commands spoken by the user 12. The audio communication system 914 may also communicate with the smart phone or any mobile communication system of the user 12, for example, via Bluetooth®. Other information may be gathered and stored and updated on a processor 918, also carried on the head support 902, such as location of user 12 (e.g., via GPS tracking) and vital characteristics of the user 12, such as pulse (heart rate), respiration rate, and body temperature. Thus, heart rate monitors, microphones, or thermometers may be utilized. The communication system may be configured to operate on an iOS device, an Android device, or an internal network. FIG. 68 illustrates an alternative embodiment of a personal protection system 935, that is similar to the personal protection system 900, but additionally has a tinted facial shield 939, which may comprise a colored or tinted plastic. This tinting is of use in situations requiring additional eye protection, for example, to protect against damage from laser light exposure. This may include healthcare or general industrial use. FIG. 69 illustrates an alternative embodiment of a personal protection system 937, that is similar to the personal protection system 900, but additionally has a facial shield 941 formed from a clear sheet 943, but having a colored or tinted cover sheet 945. The cover sheet 945 is secured to the clear sheet 943 with an adhesive backing 949, and can be peeled from the clear sheet 943 but us of a peel-away tab 947. In some embodiments, two or more cover sheets 945 may be utilized, a first cover sheet 945 adhered to the clear sheet 943, and each successive cover sheet 945 attached over the previous cover sheet 945.

FIG. 49 illustrates a personal protection system 920 comprising a head support 922 and a facial shield 924. The facial shield 924 is coupled to the head support 922 via an extender 926 that is similar to the extender 806 of FIGS. 32-35. A cuff 928 is coupled to the facial shield 924 similar to the cuff 629, 662 of the embodiments of FIGS. 28-29 and 47. The user 12 is wearing an in-lens loupe 930, connected to eyeglasses 931, which may have prescription or non-prescription lenses. The extender 926 allows for increased space between the user 12 and the facial shield 924 to easily accommodate the loupe 930, and allow adjustment of it. The user 12 may adjust the loupe 930 without touching it by keeping the cuff 928 in between the user's hand and the loupe 930. In other words, the user 12 grasps the loupe 930 using the cuff 928 as a hand cover. The loupe 930 may also utilize a light 932. The light 932 is shown in FIG. 49 as separate from the loupe 930, but alternatively or additionally, the loupe 930 itself may carry a light. The head support 922 may also carry a camera 933 (see FIG. 52).

Alternative lights 932a, 932b are illustrated in FIG. 50, carried on the extender 926, and provide beams 934a, 934b that converge onto the area of view. Thus, the pair of lights 932a, 932b maximize the intensity and optimize ability to focus on the field of view. In some embodiments, the lights 932a, 932b are each adjustable such that the beams 934a, 934b may be oriented in different areas with some or no overlap. FIG. 51 illustrates a detail of the front portion of the extender 926. The extender 926 provides several controls 936 that are easily accessible by the user 12 for operation. The controls 936 may comprise membrane switches, and may be set for on/off toggle, or for hold-down on/release off. A phone dialer 938 button may initiate a phone call, or pause, or end the call. A first connection button 940 may be configured to connect to a first person or office (e.g., administration), for two-way communication. A second connection button 942 may be configured to connect to a second person or office (e.g., reporting), or may be configured to automatically start a recording for dictating a case report. The on/off of any of these controls 936 may alternatively be voice-activated.

A light on/off button 944 is configured to allow the lights 932 to be turned on or off. In other embodiments, the button 944 may alternatively or additionally operate other lights, including the ability to dim or turn on or off the lights of the procedural room. A camera button 946 is configured to start, pause, or stop the camera 933. A recording button 948 is configured to start, pause, or stop a video and/or audio recording. Turning to FIG. 52, the camera 933 is mounted on top of the extender 926 to allow recording of a procedure as it occurs. Training material can be developed by turning the camera on at key points in the procedure and recording only the most critical elements. Corresponding audio comments can also be recorded at the same time (e.g., via an audio communication system 914). The extender 926 serves to make the head support 922 adaptable to a variety of different setups. A first setup may be incorporated using the facial shield 924 directly attached to the head support 922, without the extender 926. A second setup may be incorporated using the extender 926 between the facial shield 924 and the head support 922, for example when more space is required for accommodating a loupe 930 or other equipment or facewear/eyewear. The user 12 may be able to give voice commands to turn the camera 933 on of off of turn the lights 932 on or off, or to vary their usage in other manners (dim, brighten). In some embodiments, voice commands may be used to change the magnification of the loupe. For example, “zoom to 3X,” or “zoom to 5X,” etc.

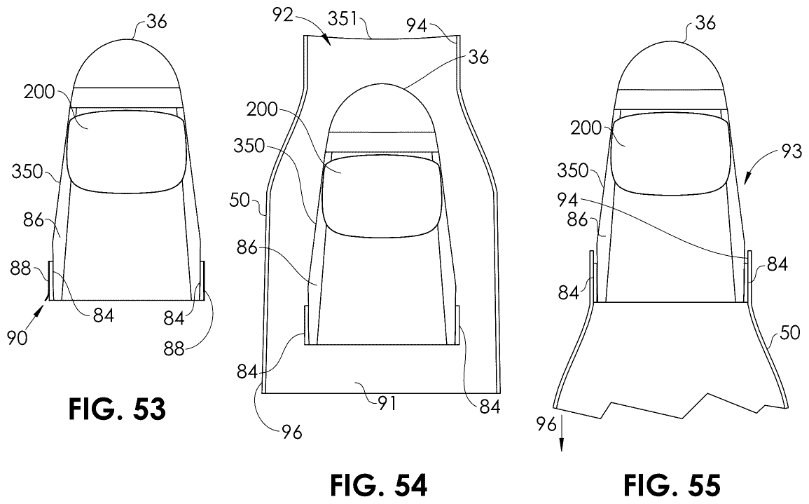

FIGS. 53-55 illustrate a hood 350 having one or more adhesive strips 84 carried on a lower portion 86 of the hood 350. The adhesive strips 84 include peel-away cover strips 88 that are shown being removed by peeling 90 in FIG. 53. A user (or an assistant) places the hood 350 over the user's head and then peels the cover strips 88 (or has them peeled by another). As shown in FIG. 54, the user, or an assistant, places a gown 50 over the hood 350 so that a first open portion 91 is slid over the top of the hood 350, and then an open upper portion 351 of the gown 50 having an opening 92 and an inner surface 94 is slid down over the hood 350. As shown in FIG. 55, the inner surface 94 is then adhered to the adhesive strips 84, thus forming a substantially continuous cover 93 from the top portion 38 of the hood 350 to the lower portion 96 of the gown 50. There are no ties that need to be tied, and the gowning process is significantly shortened by the joining of the inner surface 94 to the adhesive strips 84. The one or more adhesive strips 84 may comprise a single, substantially circumferential outer strip on the lower portion 86 of the hood 350, or may comprise a series of smaller strips arrayed around the circumference of the lower portion 86 of the hood 350. When the use of the hood 350 and the gown 50 is complete, the user or an assistant simply removes the substantially continuous cover 93 comprising the hood 350 and gown 50. This can save many standard doffing steps, because, if contaminated, the hood 350 and gown 50 can be removed without likelihood of contact with the contaminated portions. This also saves a significant amount of time because of significantly fewer steps.

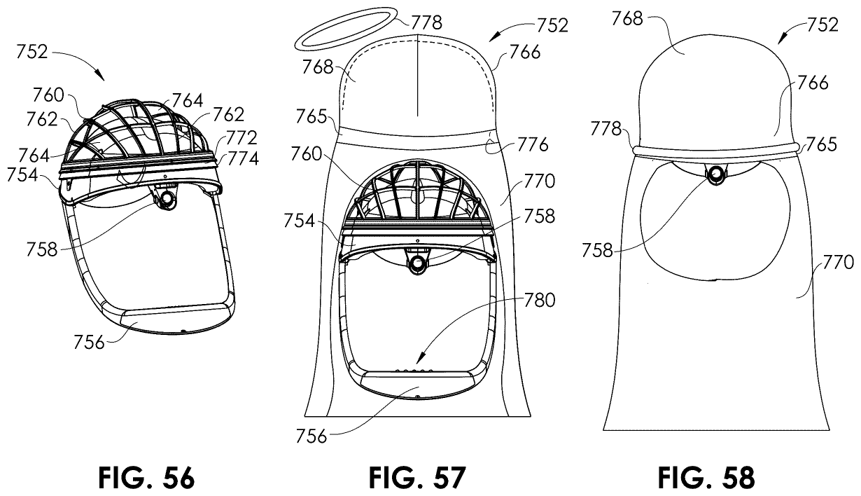

FIG. 56 illustrates a personal environmental protection system 752 comprising a support portion 754 and a chin bar 756. A light 758 is carried by the support portion 754 and is configured to provide additional illumination for a procedure performed by the user. The light 758 may comprise high intensity LEDs. A filter frame 760 is carried on the support portion 754 and includes a series of ribs 762 defining spaces 764 between the ribs 762 configured to allow the circulation of filtered air. A fan (not shown) is configured to cause the flow of air within the system 752. A filter cartridge/hood assembly 766 FIG. 57 comprises a filter cartridge 768 within a hood 770, and is configured to sealably seat on top of the filter frame 760. In FIG. 57 the filter cartridge/hood assembly 766 is placed over the support portion 754, and in FIG. 58 the filter cartridge/hood assembly 766 is sealably coupled to the filter frame 760. Returning to FIG. 56, a circumferential gutter or seat 772 extends continuously around a lip 774 of the filter frame 760. As seen in FIG. 57, an internal circumferential protrusion 776 is configured to seat into the gutter 772. In some cases, the protrusion 776 is configured to snap into the gutter 772. In alternative embodiments, the lip 774 of the filter frame 760 may have the protrusion 776 and the filter cartridge/hood assembly 766 may have the gutter 772. A circular retainer 778 (an o-ring, or alternatively, a continuous circular spring or an elastic/rubber band) is configured to be forcibly snapped around an outer portion 765 of the filter cartridge/hood assembly 766 to force the circumferential protrusion 776 into the circumferential gutter 772 and to hold the filter cartridge/hood assembly 766 and the filter frame 760 together in a sealing manner. The retainer 778 further assures that the filter cartridge 768 will not be displaced, if contacted with force. As shown in FIG. 58 once in place, the hood 770 extends vertically and is configured to cover at least the shoulder area of the user. The filter cartridge/hood assembly 766 can be removed by removing the retainer 778 and then detaching the filter cartridge/hood assembly 766 from the filter frame 760.

LEDs 780 are arrayed on the chin bar 756 and are configured to light up to notify the user or other medical personnel of particular events. For example, one or more yellow LED may be illuminated if sufficient air flow cannot be maintained. The level of battery charge may be indicated by the number of green LEDs being lit (e.g., three if fully charged, and two or one if less charged). A red LED may be illuminated if there is no charge or if very little charge less (e.g., approximately fifteen minutes remaining). The LEDs may be combined with voice control, and may show status of the procedure, as determined and described by the user/attending physician. If the light 758 comprises one or more LED itself, air flow in the personal environmental protection system 752 may further be configured (shaped, oriented) to cool the LEDs in the light 758 using forced convection.

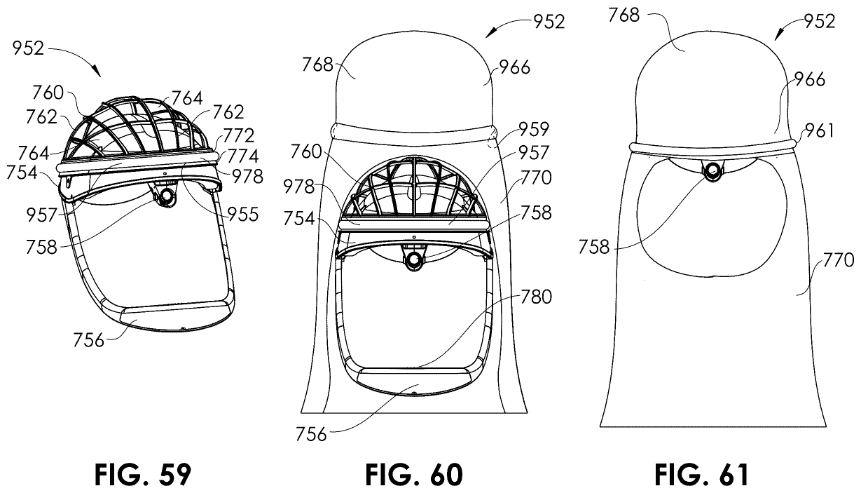

FIGS. 59-61 illustrates an alternative embodiment of a personal environmental protection system 952 that is similar to the personal environmental protection system 752, but with a reversed sealing surface. An o-ring seal 978 is carried on the circumferential gutter or seat 772. In some embodiments, the internal diameter 955 of the o-ring seal 978 may be permanently bonded to the seat 772, for example with flexible silicone or urethane adhesive. An inner circumferential surface 959 inside the filter cartridge/hood assembly 966 is configured to mate with the outer diameter 957 of the o-ring seal 978. When the o-ring seal 978 is sealingly mated with the inner circumferential surface 959, as shown in FIG. 61, an outer portion 961 of the filter cartridge/hood assembly 966 bulges out. In some embodiments, the o-ring seal 978 may instead by an elastomeric circular ridge that is directly molded or bonded onto the support portion 754.

In the embodiments represented in FIGS. 53-61, the adhesive strips 84, the circular retainer 778, or the o-ring seal 978 not only allow simpler setup, cleaning, and removal, but also provide a more secure sealing, to allow air to circulate only within desired areas.

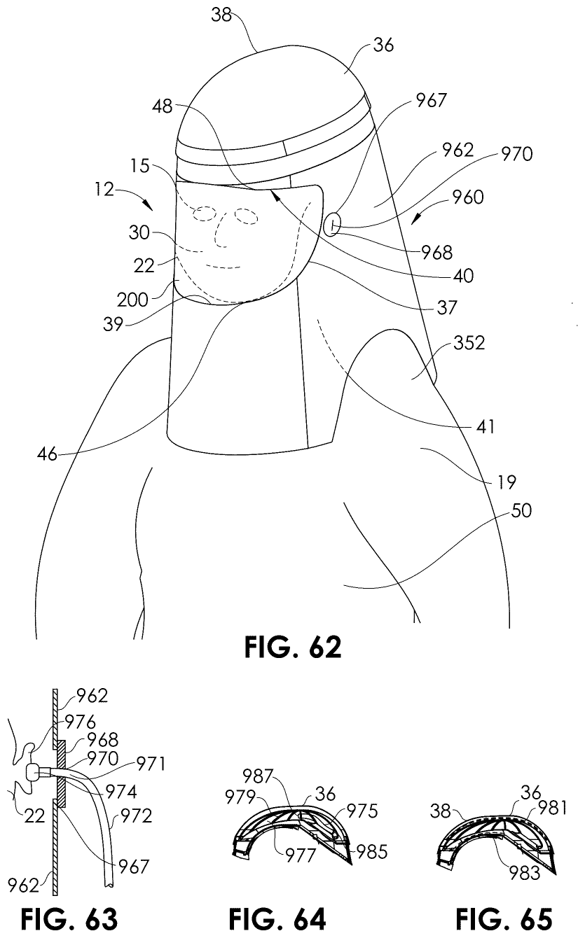

FIG. 62 illustrates a user 12 wearing an alternative personal environmental protection system 960 comprising a hood 962, which are similar to the system 34 and hood 350 of FIG. 41, however they are configured for improved acoustic characteristics. The hood 962 is secured along a circular seam 967 to a silicone circle 968 having a slit 970. In other embodiments, the seam 967 and silicone circle 968 can be non-circular shapes (square, rectangle, etc.). The silicone circle 968 may comprise a silicone sheet having a thickness of between about 0.25 cm and about 2 cm, or between about 0.50 cm and about 1 cm. The silicone may be substituted for another elastomeric or supple material, such as polyurethane or latex. The seam 967 sealingly bonds to the silicone, and the slit 970 is normally closed. As shown in FIG. 63, a shaft 971 (aural tube) of a stethoscope 972 is placed through the slit 970, which allows the passage of the earpiece 974, and which can then be slipped into the ear 976 of the user 12. The silicone distends or stretches and the slit 970 substantially seals or at least closes around the shaft 971 enough to maintain the interior of the hood 962 substantially isolated from the ambient air. Though not shown, there are two silicone circles 968 with slits 970, one on each side of the hood 962 to accommodate both shafts 971 of a bi-aural stethoscope 972. When the use of the stethoscope 972 is no longer needed, the shafts 971 and earpieces 974 may be removed, and the slits 970 substantially return to their original closed configuration. Though the silicone circle 968 is shown in FIG. 63 attached to an outer side of the hood 962 material, alternatively, it can be attached to an inner side, or be attached to both sides, or even in a flush manner.

FIG. 64 illustrates an embodiment of support portion 36 (e.g., helmet) for the personal environmental protection system 960 having curved air channels 975 within the spaces 977 between the ribs 979. The channels 975 are pre-shaped by molding, or in some embodiments may comprise tubes or baffling surfaces that are slipped in certain portions in the spaces 977. The channels 975 serve to minimize eddies or turbulent areas, and increase laminar flow, in order to lower the intensity of sound generated by the air flow. Thus, communication is improved both by the user 12 having less interfering internal sounds. Additionally, when used with the audio communication systems disclosed herein, there are fewer interfering internal sounds being picked up by an internal microphone that would be heard by others. Each channel 975 may have in inlet 985 and an outlet 987. FIG. 65 illustrates an alternative feature that may be utilized with the support portion 36 with or without the channels 975. An outer acoustic muffler 981 is incorporated within a top portion 38 of the support portion 36. The muffler 981 may comprise acoustic foam and/or acoustic adhesive. A second muffler 983 at an inner portion of the support portion 36 may also be utilized (or alternatively be utilized). The second muffler 983 may comprise an acoustic foam that is shaped to also snugly grasp the head 22 of the user 12. The channels 975 or mufflers 981, 983 can be configured to drop the intensity of the sound heard by the ears 976 of the user 12 by as much as 3 dB, or even as much as 6 dB, or between about 3 dB and about 6 dB. In some embodiments, the channels 975 may comprise silencers, which comprise a thin rigid tube having soundproofing material therein.

FIG. 66 illustrates a personal protection system 901 having a head support 16, and a facial shield 518. A blower 903 comprises a motor 905 and is configured to circulate air through a filter cartridge 907 (FIG. 67). As described herein, communication in personal protection settings can be important. In addition, users 12 may sometimes need to hear soft or precise sounds, such as those obtained via listening to a stethoscope. Thus, at times, it may be desired to quiet the noises made by forces-air systems comprising blowers 903 to better allow listening by the user 12, for auditory accuracy. The noises may include air flow and/or motor operation. A button 909 carried on the chin bar 42 comprises a switch electrically coupled to the motor 905 of the blower 903. To maintain a positive pressure across a wide range of activity levels and the accompanying varying respiration rates, PAPR systems can sometimes require airflows in excess of about 200 liters per minute. The button 909 may be depressed by a user 12 (or support personnel) to reduce the level of blower operation, and to thus lower the amount of generated noise. In some embodiments, the pressing of the button 909 reduces the output of the blower 903 to below 125 liter per minute, significantly lowering the intensity of the sound caused by the forced air. In some embodiments, the button 909 may be a three-position switch comprising the following settings: off, low, and high. In other embodiments, there may be four or more positions: off, low, medium, high.

In other embodiments, a microprocessor 911 is coupled to the button 909 and the blower 903, and is configured to run a subroutine which automatically lowers the operation of the blower (e.g., to a low speed) for a particular amount of time (e.g., five minutes, three minutes, one minute, 30 seconds, or 15 seconds). The increase to high level is then triggered automatically. In some embodiments, a sound intensity meter (dB meter) 913, coupled to the microprocessor 911, measures the sound intensity within the interior area of the head support 16, and the facial shield 518, and adjusts the power supplied to the blower 903 proportionally to lower the internal sound intensity below a target amount. In some embodiments, the microprocessor 911 comprises a logic controller. Thus, a feedback loop allows for automatic adjustment of the sound levels. The button 909 may be configured such that a first position (or toggle) causes the feedback loop to operate, while a second position (or toggle) turns off the feedback loop. In some embodiments, the button 909 may be coupled to the blower 903 and/or to the microprocessor 911 via Bluetooth® communication or other wireless means. In some embodiments, the operation may be controlled via voice control: “operate feedback loop,” “run blower on low level,” etc.

While the foregoing is directed to embodiments of the present disclosure, other and further embodiments may be devised without departing from the basic scope thereof. For example, the embodiments having communication systems (audio, visual, etc.) may incorporate any of the styles of facial shield (snap-on, sealed, tinted, etc.).

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

1-2. (canceled)

3. A personal protection system, comprising:
a support configured to engage the head of a user, the support comprising an anterior surface;
a facial shield coupled to the support;
a projector carried by the support and configured to project onto an internal surface of the facial shield; and
an extender configured to couple the facial shield to the support, the extender comprising an interior side configured to closely match the anterior surface of the support, the extender comprising a thickness configured to increase a distance between the internal surface of the facial shield and the eyes of the user.

4. The personal protection system of claim 3, wherein the interior side of the extender comprises a concavity.

5. The personal protection system of claim 4, wherein the anterior surface of the support comprises a convexity.

6. The personal protection system of claim 3, further comprising one or more hooks carried on the extender, wherein the facial shield comprises one or more holes configured to connect to the one or more hooks.

7. The personal protection system of claim 6, wherein the one or more hooks comprise a center hook.

8. The personal protection system of claim 7, wherein the one or more holes comprises a center hole, and wherein the center hook is configured to lock to the center hole.

9. The personal protection system of claim 8, wherein center hook is configured be rotated to lock to the center hole.

10. The personal protection system of claim 3, wherein the thickness of the extender is configured to increase a distance between the internal surface of the facial shield and the nose of the user.

11. The personal protection system of claim 3, wherein the thickness of the extender is configured to increase a distance between the internal surface of the facial shield and the mouth of the user.

12. The personal protection system of claim 3, further comprising a user-operatable control carried on the extender.

13. A personal protection system, comprising:
a support configured to engage the head of a user, the support comprising an anterior surface;
a facial shield coupled to the support;
a projector carried by the support and configured to project onto an internal surface of the facial shield; and
an extender configured to couple the facial shield to the support, the extender comprising an interior side configured to closely match the anterior surface of the support, the extender comprising a thickness configured to increase a volume between the internal surface of the facial shield and the face of the user.

14. The personal protection system of claim 13, wherein the interior side of the extender comprises a concavity.

15. The personal protection system of claim 14, wherein the anterior surface of the support comprises a convexity.

16. The personal protection system of claim 13, further comprising one or more hooks carried on the extender, wherein the facial shield comprises one or more holes configured to connect to the one or more hooks.

17. The personal protection system of claim 16, wherein the one or more hooks comprise a center hook.

18. The personal protection system of claim 17, wherein the one or more holes comprises a center hole, and wherein the center hook is configured to lock to the center hole.

19. The personal protection system of claim 18, wherein center hook is configured be rotated to lock to the center hole.

20. The personal protection system of claim 13, wherein the thickness of the extender is configured to increase a distance between the internal surface of the facial shield and the nose of the user.

21. The personal protection system of claim 13, wherein the increased volume is configured to facilitate air flow to reduce fogging of the facial shield.

22. The personal protection system of claim 13, wherein the increased volume is configured to facilitate air flow to reduce an increase in an internal carbon dioxide level.
